(19) United States
(12) Patent Application Publication
(10) Pub. No.: US 2016/0362424 A1
(43) Pub. Date: Dec. 15, 2016

(54) SALTS OF (S)-7-(1-(9H-PURIN-6-YLAMINO)ETHYL)-6-(3-FLUOROPHENYL)-3-METHYL-5H-THIAZOLO[3,2-A]PYRIMIDIN-5-ONE

(71) Applicant: Incyte Corporation, Wilmington, DE (US)

(72) Inventors: Yun-Long Li, Chadds Ford, PA (US); Brian W. Metcalf, Moraga, CA (US); Hui-Yin Li, Hockessin, DE (US)

(21) Appl. No.: 15/150,966

(22) Filed: May 10, 2016

Related U.S. Application Data
(60) Provisional application No. 62/159,760, filed on May 11, 2015.

(51) Int. Cl. C07D 519/00; A61K 45/06; C07C 65/03; A61K 31/52; C07C 55/14; C07C 309/04

(52) U.S. Cl. CPC C07D 519/00; C07C 55/14; C07C 309/04; C07C 65/03; A61K 31/52; A61K 45/06; C07B 2200/13

(57) ABSTRACT

The present invention relates to salt forms of the phosphoinositide 3-kinase (PI3K) inhibitor (S)-7-(1-(9H-purin-6-ylamino)ethyl)-6-(3-fluorophenyl)-3-methyl-5H-thiazolo[3,2-a]pyrimidin-5-one, pharmaceutical compositions comprising the same, and methods of using the salts and compositions for the treatment of PI3K-associated diseases such as cancer.

### FIELD OF THE INVENTION

The present invention relates to salt forms of the phosphoinositide 3-kinase (PI3K) inhibitor (S)-7-(1-(9H-purin-6-ylamino)ethyl)-6-(3-fluorophenyl)-3-methyl-5H-thiazolo[3,2-a]pyrimidin-5-one, pharmaceutical compositions comprising the same, and methods of using the salts and compositions for the treatment of PI3K-associated diseases such as cancer.

### BACKGROUND OF THE INVENTION

The compound (S)-7-(1-(9H-purin-6-ylamino)ethyl)-6-(3-fluorophenyl)-3-methyl-5H-thiazolo[3,2-a]pyrimidin-5-one having Formula I:

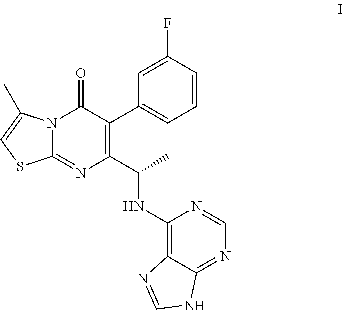 [SMILES: Cc1csc2nc([C@H](C)Nc3ncnc4[nH]cnc34)c(-c3cccc(F)c3)c(=O)n12]

is a phosphoinositide 3-kinase (PI3K) inhibitor. The compound of Formula I, as well as its preparation and use, have been described in U.S. Publication No. 2011/0015212, which is incorporated herein by reference in its entirety. For the manufacture, purification, and formulation of a drug, it is typically advantageous to employ a form of the drug having superior stability or other desirable formulation property exhibited by, for example, one or more salt or crystalline forms of the drug. Accordingly, the salt forms of the compound of Formula I provided herein help satisfy the ongoing need for new forms of PI3K inhibitors.

### SUMMARY OF THE INVENTION

The present invention provides the adipic acid, hydrochloric acid, methanesulfonic acid, gentisic acid, or ethanesulfonic acid salt forms of the compound of Formula I:

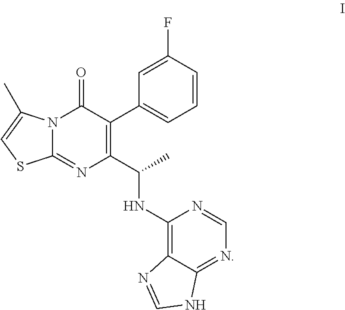 [SMILES: Cc1csc2nc([C@H](C)Nc3ncnc4[nH]cnc34)c(-c3cccc(F)c3)c(=O)n12]

The present invention further provides a composition comprising one or more salt forms of the present invention and a pharmaceutically acceptable carrier.

The present invention further provides a method of treating a disease associated with expression or activity of a PI3K in a patient comprising administering to the patient a therapeutically effective amount of a salt form of the invention.

### BRIEF DESCRIPTION OF THE DRAWINGS

FIG. 1 depicts an X-ray powder diffraction pattern for a crystalline adipic acid salt form of the compound of Formula I.

FIG. 2 depicts a differential scanning calorimetry (DSC) thermogram for a crystalline adipic acid salt form of the compound of Formula I.

FIG. 3 depicts a thermogravimetric analysis (TGA) thermogram for a crystalline adipic acid salt form of the compound of Formula I.

FIG. 4 depicts an X-ray powder diffraction pattern for a crystalline hydrochloric acid salt form of the compound of Formula I.

FIG. 5 depicts a differential scanning calorimetry (DSC) thermogram for a crystalline hydrochloric acid salt form of the compound of Formula I.

FIG. 6 depicts a thermal gravimetric analysis (TGA) thermogram for a crystalline hydrochloric acid salt form of the compound of Formula I.

FIG. 7 depicts an X-ray powder diffraction pattern for a crystalline methanesulfonic acid salt form of the compound of Formula I.

FIG. 8 depicts a differential scanning calorimetry (DSC) thermogram for a crystalline methanesulfonic acid salt form of the compound of Formula I.

FIG. 9 depicts a thermal gravimetric analysis (TGA) thermogram for a crystalline methanesulfonic acid salt form of the compound of Formula I.

FIG. 10 depicts an X-ray powder diffraction pattern for a crystalline gentisic acid salt form of the compound of Formula I.

FIG. 11 depicts a differential scanning calorimetry (DSC) thermogram for a crystalline gentisic acid salt form of the compound of Formula I.

FIG. 12 depicts a thermal gravimetric analysis (TGA) thermogram for a crystalline gentisic acid salt form of the compound of Formula I.

FIG. 13 depicts an X-ray powder diffraction pattern for a crystalline ethanesulfonic acid salt form of the compound of Formula I.

FIG. 14 depicts the ¹H NMR (400 MHz, d₆-DMSO) spectrum for the adipic acid salt of the compound of Formula I.

FIG. 15 depicts the ¹H NMR (400 MHz, d₆-DMSO) spectrum for the methanesulfonic acid salt of the compound of Formula I.

FIG. 16 depicts the ¹H NMR (400 MHz, d₆-DMSO) spectrum for the gentisic acid salt of the compound of Formula I.

### DETAILED DESCRIPTION

The present invention provides pharmaceutically acceptable salt forms of (S)-7-(1-(9H-purin-6-ylamino)ethyl)-6-(3-fluorophenyl)-3-methyl-5H-thiazolo[3,2-a]pyrimidin-5-one having Formula I:

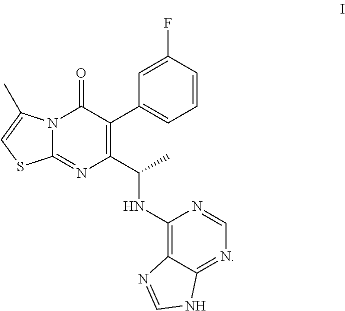 [SMILES: Cc1csc2nc([C@H](C)Nc3ncnc4[nH]cnc34)c(-c3cccc(F)c3)c(=O)n12]

In some embodiments, the salt form of the compound of Formula I is the adipic acid salt form.

In some embodiments, the salt form of the compound of Formula I is the hydrochloric acid salt form.

In some embodiments, the salt form of the compound of Formula I is the methanesulfonic acid salt form.

In some embodiments, the salt form of the compound of Formula I is the gentisic acid salt form.

In some embodiments, the salt form of the compound of Formula I is the ethanesulfonic acid salt form.

In some embodiments, salts of the invention can be prepared by any suitable method for the preparation of acid addition salts. For example, the free base compound of Formula I can be combined with the desired acid in a solvent or in a melt. Alternatively, an acid addition salt of Formula I can be converted to a different acid addition salt by anion exchange. Salts of the invention which are prepared in a solvent system can be isolated by precipitation from the solvent. Precipitation and/or crystallization can be induced, for example, by evaporation, reduction of temperature, addition of anti-solvent, or combinations thereof.

In some embodiments, the salts of the invention are crystalline, including crystalline forms which are anhydrous, hydrated, non-solvated, or solvated. Example hydrates include hemihydrates, monohydrates, dihydrates, and the like. In some embodiments, the crystalline salts are anhydrous and non-solvated. By “anhydrous” is meant that the crystalline salt contains no bound water in the crystal lattice structure, i.e., the compound does not form a crystalline hydrate.

In some embodiments, the salts of the invention are substantially isolated. By “substantially isolated” is meant that the salt is at least partially or substantially separated from the environment in which it was formed or detected. Partial separation can include, for example, a composition enriched in the salt of the invention. Substantial separation can include compositions containing at least about 50%, at least about 60%, at least about 70%, at least about 80%, at least about 90%, at least about 95%, at least about 97%, or at least about 99% by weight of the salt.

Salts of the invention also include all isotopes of atoms occurring in the salts. Isotopes include those atoms having the same atomic number but different mass numbers. For example, isotopes of hydrogen include tritium and deuterium.

The salt forms of the invention were found to be highly crystalline, a desirable property which would facilitate, for example, purification of the drug such as by crystallization and recrystallization as necessary. Further, a crystalline form tends to be more stable and can be easier to mill or micronize when formulating a drug. Crystalline salts also tend have excellent properties with respect to solubility and are usually more suitable to be manufactured reproducibly in a clear acid/base ratio, facilitating the preparation of liquid formulations for oral as well as for intravenous applications.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

Crystalline forms are most commonly characterized by XRPD. An XRPD pattern of reflections (peaks) is typically considered a fingerprint of a particular crystalline form. It is well known that the relative intensities of the XRPD peaks can widely vary depending on, inter alfa, the sample preparation technique, crystal size distribution, filters, the sample mounting procedure, and the particular instrument employed. In some instances, new peaks may be observed or existing peaks may disappear, depending on the type of instrument or the settings (for example, whether a Ni filter is used or not). As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” as used in the context of XRPD herein is meant to encompass the above-mentioned variations.

In the same way, temperature readings in connection with DSC, TGA, or other thermal experiments can vary about ±4° C. depending on the instrument, particular settings, sample preparation, etc. For example, with DSC it is known that the temperatures observed will depend on the rate of the temperature change as well as the sample preparation technique and the particular instrument employed. Thus, the values reported herein related to DSC thermograms can vary, as indicated above, by ±4° C. Accordingly, a crystalline form reported herein having a DSC thermogram “substantially” as shown in any of the Figures is understood to accommodate such variation.

### Adipic Acid Salt

The adipic acid salt of the compound of Formula I can be prepared by any suitable method for preparation of adipic acid addition salts. For example, the compound of Formula I can be combined with adipic acid (e.g., about 1.0 eq or more) in a crystallizing solvent and the resulting salt can be isolated by filtering the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains methanol, ethanol, 1-propanol, or isopropanol. In some embodiments, the alcohol contains isopropanol. In some embodiment, the solvent contains a mixture of water, alcohol and ketone. Suitable ketones include acetone, methyl ethyl ketone, diethylketone, methyl isobutyl ketone, and the like. In some embodiments, the ketone is methyl isobutyl ketone.

In some embodiments, the crystallizing solvent is an alcohol such as isopropanol. In other embodiments, the crystalizing solvent contains water and alcohol in a volume ratio of about 1:2 to about 1:20, about 1:5 to about 1:12, or about 1:9.

In some embodiments, the crystallizing solvent is heated to a temperature of at least about 50° C. to induce the crystallization at a practical rate. In some embodiments, a temperature from about 50° C. to about 80° C. is used. In some embodiments, a temperature of about 50° C., about 55° C., about 60° C., about 65° C., about 70° C., about 75° C. or about 80° C. is used.

In some embodiments, crystallization will be complete within about 24 to about 72 hours, but longer and shorter periods are possible depending on the choice of crystallizing solvent and temperature.

The precipitation and/or crystallization of the adipic acid salt, in some embodiments, is carried out by filtering the salt from solution. In other embodiments of the invention, the precipitation and/or crystallization is induced by the addition of anti-solvent. A suitable anti-solvent can contain any solvent in which the salt is poorly soluble such as a ketone (e.g., methyl isobutyl ketone).

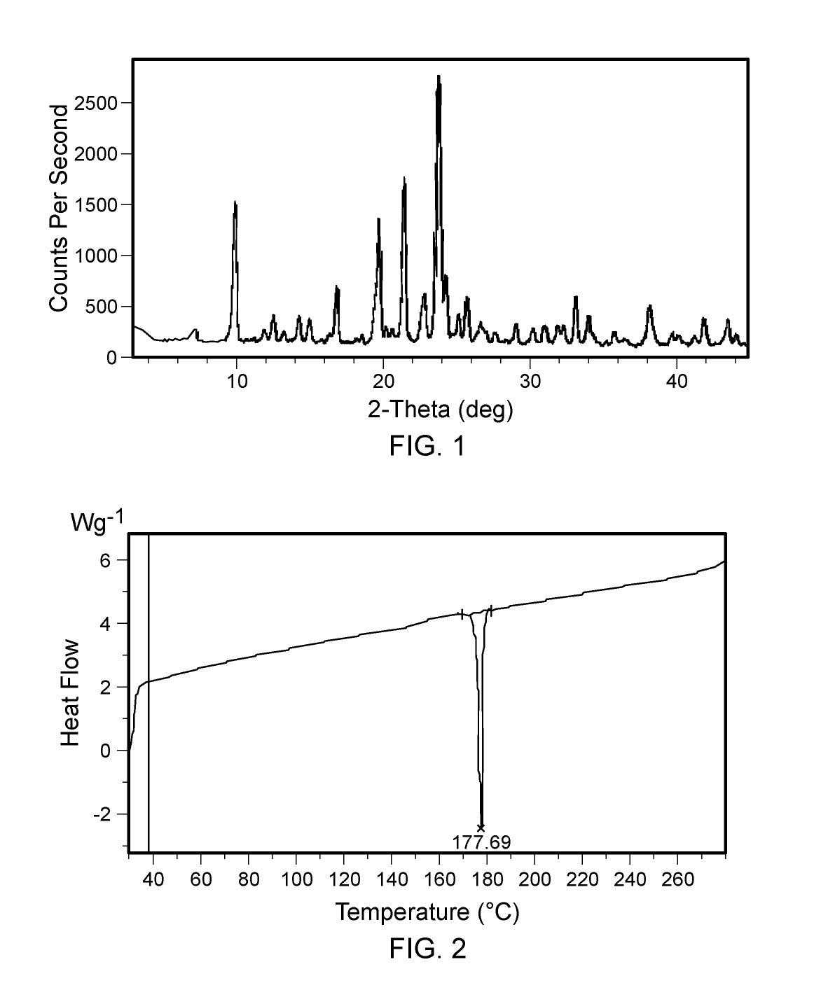

Crystalline adipic acid salt forms of the compound of Formula I can be identified by their unique signatures with respect to, for example, X-ray powder diffraction, differential scanning calorimetry (DSC), thermogravimetric analysis (TGA), and solid state NMR. In some embodiments, the crystalline adipic acid salt can be characterized by the X-ray powder diffraction (XRPD) pattern substantially as shown in FIG. 1. Peaks from the XRPD pattern are listed below in Table 1. The term “substantially” in this instance indicates that 2-theta values for individual peaks can vary by about ±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline adipic acid salt form of the compound of Formula I has an XRPD pattern having at least 3 peaks, in terms of 2θ, selected from Table 1 (CPS less than 1000=“+;” CPS of 1000 to 1500=“++;” CPS greater than 1500=“+++”). In some embodiments, the adipic acid salt has an XRPD pattern having at least three peaks, in terms of 2θ selected from about 9.9°, about 11.9°, about 12.6°, about 13.3°, about 14.3°, about 15.0°, about 16.5°, about 16.9°, about 19.8°, about 20.7°, about 21.5°, about 22.8°, or about 23.8°. In some embodiments, the adipic acid salt has an XRPD pattern having peaks, in terms of 2θ, at about 21.5° and about 23.8°. In some embodiments, the adipic acid salt has an XRPD pattern having peaks, in terms of 2θ, at about 9.9°, about 21.5° and about 23.8°. In some embodiments, the adipic acid salt has an XRPD pattern having peaks, in terms of 2θ, at about 9.9°, about 19.8°, about 21.5° and about 23.8°.

TABLE 1

| Observed Peak, | Intensity |
|---|---|
| 2θ (°) | (CPS) |
| 9.9 | ++ |
| 11.9 | + |
| 12.6 | + |
| 13.3 | + |
| 14.3 | + |
| 15.0 | + |
| 16.5 | + |
| 16.9 | + |
| 19.8 | ++ |
| 20.7 | + |
| 21.5 | +++ |
| 22.8 | + |
| 23.8 | +++ |
| 24.3 | + |
| 25.2 | + |
| 25.7 | + |
| 26.7 | + |
| 29.1 | + |
| 30.2 | + |
| 31.0 | + |
| 32.0 | + |
| 32.3 | + |
| 33.1 | + |
| 34.0 | + |
| 38.2 | + |
| 41.2 | + |
| 41.9 | + |
| 43.5 | + |
| 44.1 | + |

The crystalline adipic acid salt can also be identified by the DSC trace substantially as shown in FIG. 2 having, an onset temperature of an endotherm at about 176° C. and a peak at about 177° C. The term “substantially” in this instance indicates that features such as endotherms, exotherms, baseline shifts, etc. can vary about ±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

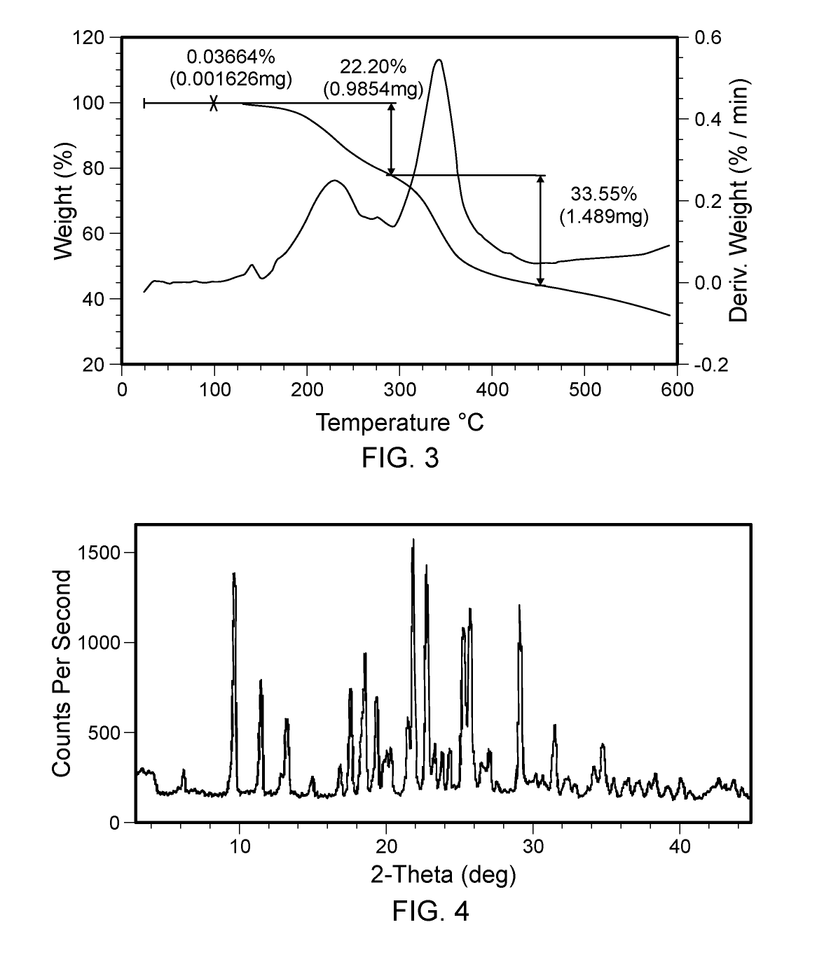

In some embodiments, the crystalline adipic acid salt has a TGA trace substantially as shown in FIG. 3.

### Hydrochloric Acid Salt

The hydrochloric acid salt of the compound of Formula I can be prepared by any suitable method for preparation of hydrochloric acid addition salts. For example, the compound of Formula I can be combined with hydrochloric acid (e.g., about 1.0 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains isopropanol.

In some embodiments, the crystallizing solvent is an alcohol such as isopropanol. In other embodiments, the crystalizing solvent contains water and alcohol in a volume ratio of about 1:2 to about 1:20, about 1:5 to about 1:12, or about 1:9.

In some embodiments, the crystallizing solvent is heated to a temperature of at least about 50° C. to induce the crystallization at a practical rate. In some embodiments, a temperature from about 50° C. to about 80° C. is used. In some embodiments, a temperature of about 50° C., about 55° C., about 60° C., about 65° C., about 70° C., about 75° C. or about 80° C. is used.

In some embodiments, crystallization will be complete within about 24 to about 72 hours, but longer and shorter periods are possible depending on the choice of crystallizing solvent and temperature.

The precipitation and/or crystallization of the hydrochloric acid salt, in some embodiments, is carried out by filtering the salt from solution. In other embodiments of the invention, the precipitation and/or crystallization is induced by the addition of anti-solvent. A suitable anti-solvent can contain any solvent in which the salt is poorly soluble such as a ketone (e.g., methyl isobutyl ketone).

The crystalline hydrochloric acid salt of the compound of Formula I can be identified by its unique signatures with respect to, for example, X-ray powder diffraction, differential scanning calorimetry (DSC), thermogravimetric analysis (TGA), and solid state NMR. In some embodiments, the crystalline hydrochloric acid salt can be characterized by the X-ray powder diffraction (XRPD) pattern substantially as shown in FIG. 4. Peaks from the XRPD are listed below in Table 2. The term “substantially” in this instance indicates that 2-theta values for individual peaks can vary by about ±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline hydrochloric salt form of the compound of Formula I has an XRPD pattern having at least 3 peaks, in terms of 20, selected from Table 2 (CPS less than 1000=“+;” CPS of 1000 to 1500=“++;” CPS greater than 1500=“+++”). In some embodiment, the hydrochloric salt has an XRPD pattern having at least 3 peaks, in terms of 2θ, at about 9.6°, about 11.4°, about 13.2°, about 16.8°, about 17.5°, about 18.5°, about 19.3°, about 20.0°, about 20.2°, about 21.5°, about 21.9°, or about 22.8°. In some embodiments, the hydrochloric acid salt has an XRPD pattern having at least one peak, in terms of 2θ, at about 9.6°. In some embodiments, the hydrochloric acid salt has an XRPD pattern having peaks, in terms of 2θ, at about 9.6°, about 21.9°, and about 22.8°. In some embodiments, the hydrochloric acid salt has an XRPD pattern having peaks, in terms of 2θ, at about 9.6°, about 21.9°, about 22.8°, about 25.7°, and about 29.1°.

TABLE 2

| Observed Peak, | Intensity |
|---|---|
| 2θ (°) | (CPS) |
| 9.6 | ++ |
| 11.4 | + |
| 13.2 | + |
| 16.8 | + |
| 17.5 | + |
| 18.5 | + |
| 19.3 | + |
| 20.0 | + |
| 20.2 | + |
| 21.5 | + |
| 21.9 | ++ |
| 22.8 | ++ |
| 23.3 | + |
| 23.8 | + |
| 24.3 | + |
| 25.3 | + |
| 25.7 | ++ |
| 26.6 | + |
| 27.0 | + |
| 27.5 | + |
| 29.1 | ++ |
| 30.1 | + |
| 30.7 | + |
| 31.5 | + |
| 32.3 | + |
| 34.2 | + |
| 34.7 | + |
| 35.5 | + |
| 44.4 | + |

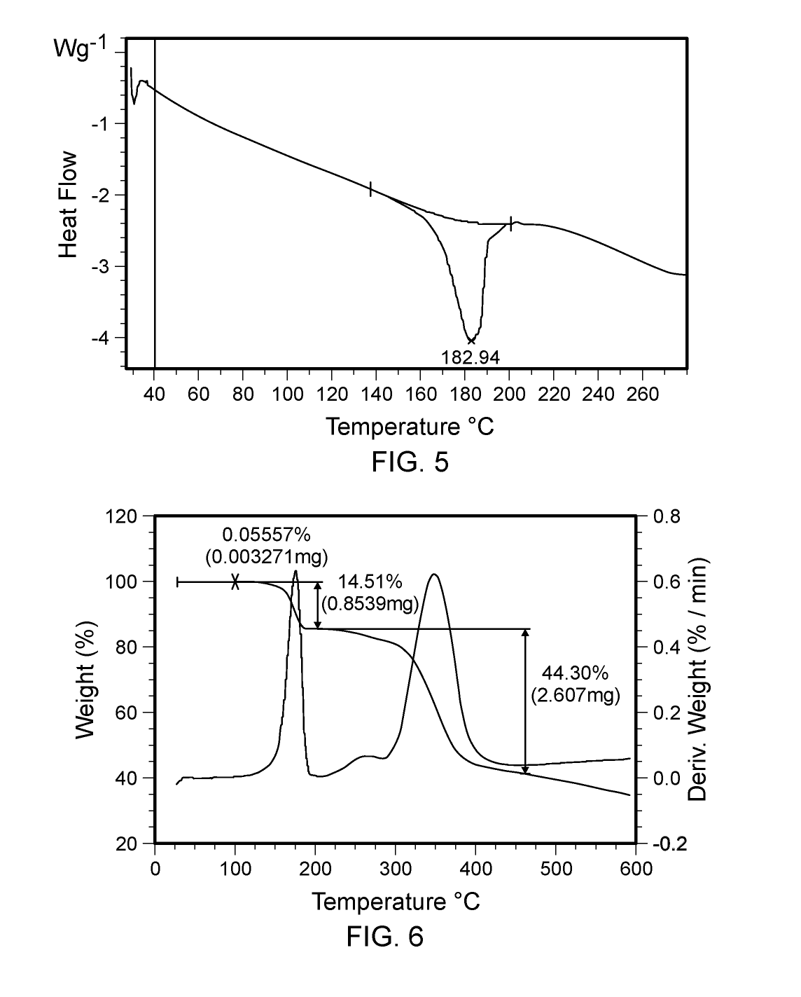

The crystalline hydrochloric acid salt can also be identified by the DSC trace substantially as shown in FIG. 5 having, as a prominent feature, an onset temperature at about 168° C. and a peak at 183° C. The term “substantially” in this instance indicates that features such as endotherms, exotherms, baseline shifts, etc. can vary about ±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

In some embodiments, the crystalline hydrochloric acid salt has a TGA trace substantially as shown in FIG. 6, exhibiting about 0.056% weight loss up to about 100° C.

### Methanesulfonic Acid Salt

The methanesulfonic acid salt of the compound of Formula I can be prepared by any suitable method for preparation of methanesulfonic acid addition salts. For example, the compound of Formula I can be combined with methanesulfonic acid (e.g., about 1.0 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains isopropanol.

In some embodiments, the crystallizing solvent is an alcohol such as isopropanol. In other embodiments, the crystalizing solvent contains water and alcohol in a volume ratio of about 1:2 to about 1:20, about 1:5 to about 1:12, or about 1:9.

In some embodiments, the crystallizing solvent is heated to a temperature of at least about 50° C. to induce the crystallization at a practical rate. In some embodiments, a temperature from about 50° C. to about 80° C. is used. In some embodiments, a temperature of about 50° C., about 55° C., about 60° C., about 65° C., about 70° C., about 75° C. or about 80° C. is used.

In some embodiments, crystallization will be complete within about 24 to about 72 hours, but longer and shorter periods are possible depending on the choice of crystallizing solvent and temperature.

The precipitation and/or crystallization of the methanesulfonic acid salt, in some embodiments, is carried out by filtering the salt from solution. In other embodiments of the invention, the precipitation and/or crystallization is induced by the addition of anti-solvent. A suitable anti-solvent can contain any solvent in which the salt is poorly soluble such as a ketone (e.g., methyl isobutyl ketone).

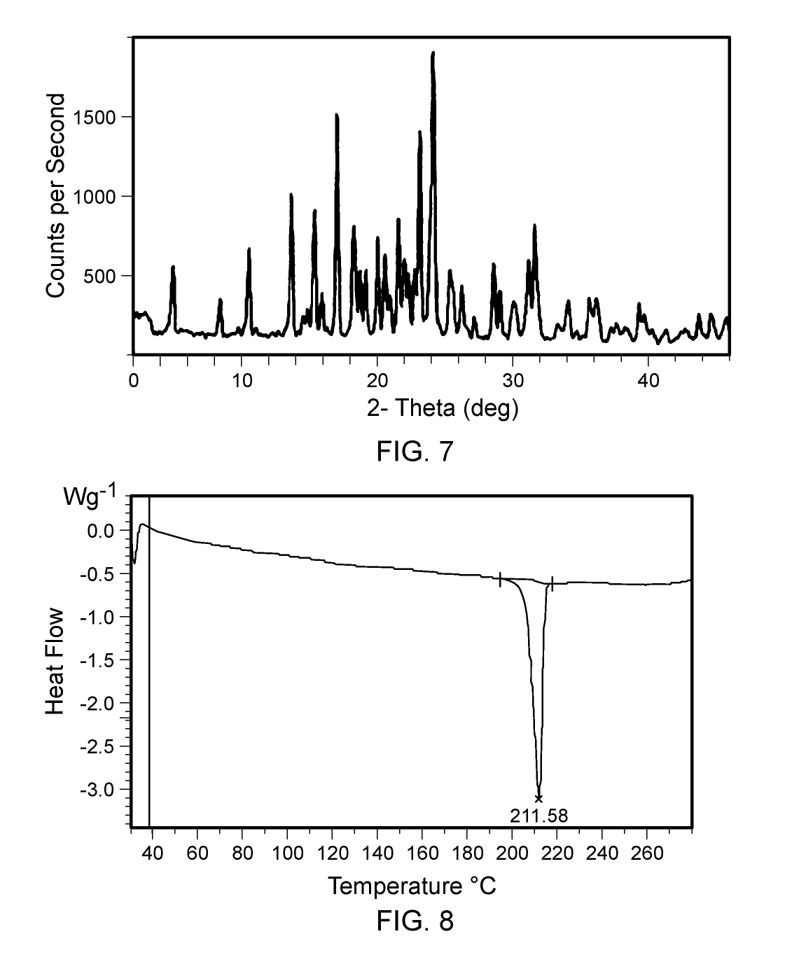

The crystalline methanesulfonic acid salt of the compound of Formula I can be identified by its unique signatures with respect to, for example, X-ray powder diffraction, differential scanning calorimetry (DSC), thermogravimetric analysis (TGA), and solid state NMR. In some embodiments, the crystalline methanesulfonic acid salt can be characterized by the X-ray powder diffraction (XRPD) pattern substantially as shown in FIG. 7. Major peaks from the XRPD are listed below in Table 3. The term “substantially” in this instance indicates that 2-theta values for individual peaks can vary by about ±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline methanesulfonic acid salt form of the compound of Formula I has an XRPD pattern having at least 3 peaks, in terms of 2θ, selected from Table 3 (CPS less than 1000=“+;” CPS of 1000 to 1500=“++;” CPS greater than 1500=“+++”). In some embodiments, the methanesulfonic acid salt has an XRPD pattern having at least 3 peaks, in terms of 2θ, selected from about 3.9°, about 5.8°, about 9.0°, about 10.2°, about 11.0°, about 14.0°, about 15.0°, about 15.6°, about 16.1°, about 17.2°, about 18.3°, about 18.7°, about 19.2°, about 20.0°, about 20.5°, about 21.4°, about 21.9°, about 22.9°, or about 23.8°. In some embodiments, the methanesulfonic acid salt has an XRPD pattern having at least one peak, in terms of 2θ, at about 17.2°. In some embodiments, the methanesulfonic acid salt has an XRPD pattern having peaks, in terms of 2θ, at about 17.2° and about 22.9°. In some embodiments, the methanesulfonic acid salt has an XRPD pattern having peaks, in terms of 2θ, at about 17.2°, about 22.9°, and about 23.8°.

TABLE 3

| Observed Peak, | Intensity |
|---|---|
| 2θ (°) | (CPS) |
| 3.9 | + |
| 5.8 | + |
| 9.0 | + |
| 10.2 | + |
| 11.0 | + |
| 14.0 | + |
| 15.0 | + |
| 15.6 | + |
| 16.1 | + |
| 17.2 | ++ |
| 18.3 | + |
| 18.7 | + |
| 19.2 | + |
| 20.0 | + |
| 20.5 | + |
| 21.4 | + |
| 21.9 | + |
| 22.9 | ++ |
| 23.8 | +++ |
| 25.1 | + |
| 25.8 | + |
| 26.7 | + |
| 28.0 | + |
| 28.5 | + |
| 29.4 | + |
| 30.5 | + |
| 30.9 | + |
| 32.5 | + |
| 33.2 | + |
| 33.8 | + |
| 34.7 | + |
| 35.2 | + |
| 38.2 | + |
| 38.4 | + |
| 41.4 | + |
| 42.4 | + |
| 43.2 | + |
| 44.3 | + |

The crystalline methanesulfonic acid salt can also be identified by the DSC trace substantially as shown in FIG. 8 having, as a prominent feature, an onset temperature at about 206° C. and a peak at about 212° C. The term “substantially” in this instance indicates that features such as endotherms, exotherms, baseline shifts, etc. can vary about ±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

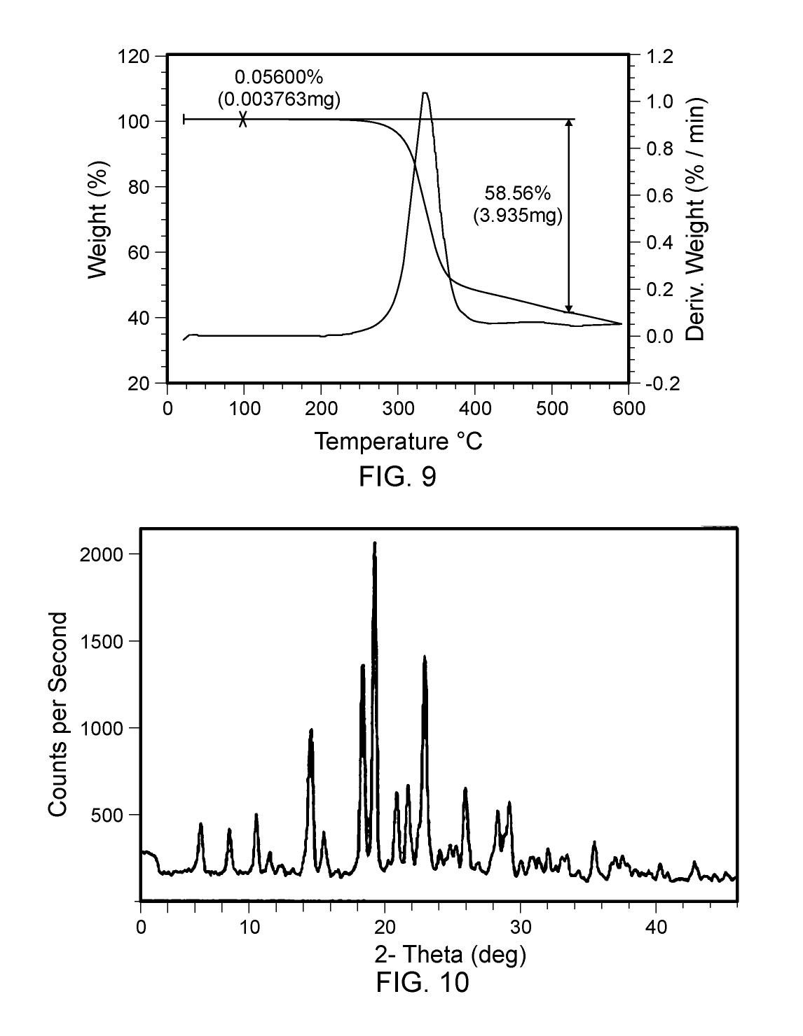

In some embodiments, the crystalline methanesulfonic acid salt has a TGA trace substantially as shown in FIG. 9, exhibiting about 0.056% weight loss up to about 100° C.

### Gentisic Acid Salt

The gentisic acid salt of the compound of Formula I can be prepared by any suitable method for preparation of gentisic acid addition salts. For example, the compound of Formula I can be combined with gentisic acid (e.g., about 1.0 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains isopropanol.

In some embodiments, the crystallizing solvent is an alcohol such as isopropanol. In other embodiments, the crystalizing solvent contains water and alcohol in a volume ratio of about 1:2 to about 1:20, about 1:5 to about 1:12, or about 1:9.

In some embodiments, the crystallizing solvent is heated to a temperature of at least about 50° C. to induce the crystallization at a practical rate. In some embodiments, a temperature from about 50° C. to about 80° C. is used. In some embodiments, a temperature of about 50° C., about 55° C., about 60° C., about 65° C., about 70° C., about 75° C. or about 80° C. is used.

In some embodiments, crystallization will be complete within about 24 to about 72 hours, but longer and shorter periods are possible depending on the choice of crystallizing solvent and temperature.

The precipitation and/or crystallization of the gentisic acid salt, in some embodiments, is carried out by filtering the salt from solution. In other embodiments of the invention, the precipitation and/or crystallization is induced by the addition of anti-solvent. A suitable anti-solvent can contain any solvent in which the salt is poorly soluble such as a ketone (e.g., methyl isobutyl ketone).

The crystalline gentisic acid salt of the compound of Formula I can be identified by its unique signatures with respect to, for example, X-ray powder diffraction, differential scanning calorimetry (DSC), thermogravimetric analysis (TGA), and solid state NMR. In some embodiments, the crystalline gentisic acid salt can be characterized by the X-ray powder diffraction (XRPD) pattern substantially as shown in FIG. 10. Major peaks from the XRPD are listed below in Table 4. The term “substantially” in this instance indicates that 2-theta values for individual peaks can vary by about ±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline gentisic acid salt form of the compound of Formula I has an XRPD pattern having at least 3 peaks, in terms of 2θ, selected from Table 4 (CPS less than 1000=“+;” CPS of 1000 to 1500=“++;” CPS greater than 1500=“+++”). In some embodiments, the gentisic acid salt has an XRPD pattern having at least 3 peaks, in terms of 2θ, selected from about 7.3°, about 9.3°, about 11.2°, about 12.1°, about 12.9°, about 14.9°, about 15.9°, about 18.6°, about 19.4°, about 20.9°, about 21.7°, or about 22.9°. In some embodiments, the gentisic acid salt has an XRPD pattern having at least one peak, in terms of 2θ, at about 18.6°. In some embodiments, the gentisic acid salt has an XRPD pattern having peaks, in terms of 2θ, at about 18.6° and about 19.4°. In some embodiments, the gentisic acid salt has an XRPD pattern having peaks, in terms of 2θ, at about 18.6°, about 19.4°, and about 22.9°.

TABLE 4

| Observed Peak, | Intensity |
|---|---|
| 2θ (°) | (CPS) |
| 7.3 | + |
| 9.3 | + |
| 11.2 | + |
| 12.1 | + |
| 12.9 | + |
| 14.9 | + |
| 15.9 | + |
| 18.6 | ++ |
| 19.4 | +++ |
| 20.9 | + |
| 21.7 | + |
| 22.9 | ++ |
| 23.9 | + |
| 24.7 | + |
| 25.0 | + |
| 25.7 | + |
| 26.6 | + |
| 28.0 | + |
| 28.8 | + |
| 29.6 | + |
| 30.4 | + |
| 30.8 | + |
| 31.5 | + |
| 32.5 | + |
| 34.7 | + |

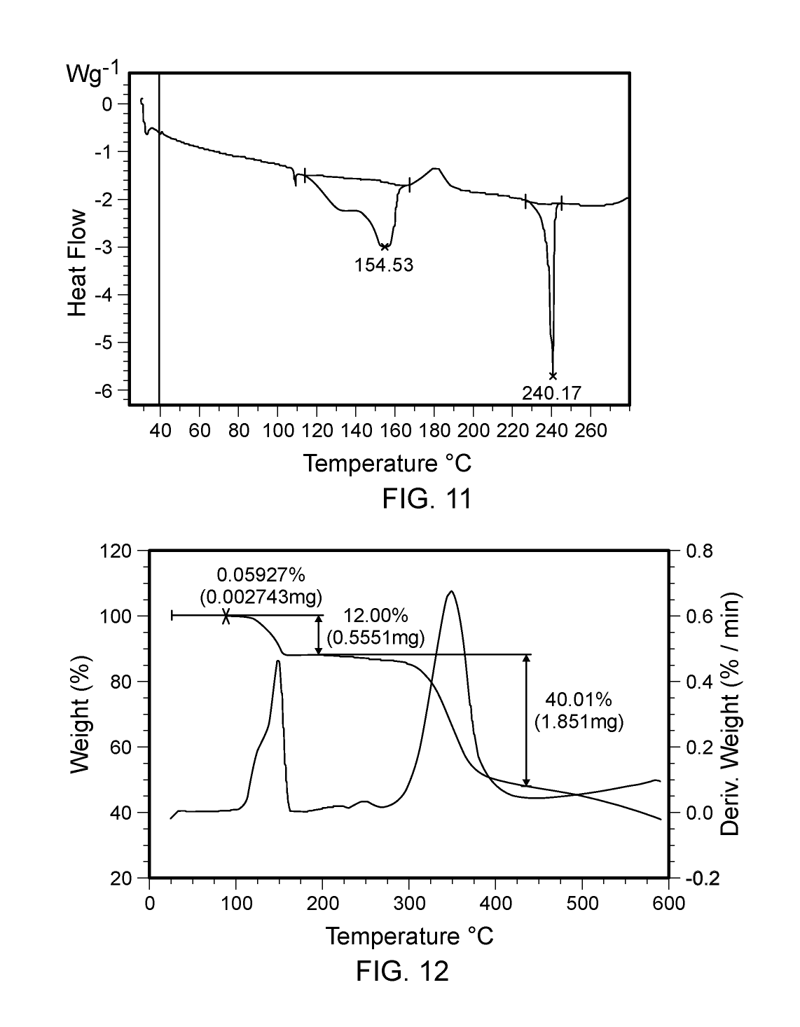

The crystalline gentisic acid salt can also be identified by the DSC trace substantially as shown in FIG. 11 having, as a prominent feature, an onset temperature at about 238° C. and a peak at about 240° C. The term “substantially” in this instance indicates that features such as endotherms, exotherms, baseline shifts, etc. can vary about ±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

In some embodiments, the crystalline gentisic acid salt has a TGA trace substantially as shown in FIG. 12, exhibiting about 0.059% weight loss up to about 100° C.

### Ethanesulfonic Acid Salt

The ethanesulfonic acid salt of the compound of Formula I can be prepared by any suitable method for preparation of ethanesulfonic acid addition salts. For example, the compound of Formula I can be combined with ethanesulfonic acid (e.g., about 1.0 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains isopropanol.

In some embodiments, the crystallizing solvent is an alcohol such as isopropanol. In other embodiments, the crystalizing solvent contains water and alcohol in a volume ratio of about 1:2 to about 1:20, about 1:5 to about 1:12, or about 1:9.

In some embodiments, the crystallizing solvent is heated to a temperature of at least about 50° C. to induce the crystallization at a practical rate. In some embodiments, a temperature from about 50° C. to about 80° C. is used. In some embodiments, a temperature of about 50° C., about 55° C., about 60° C., about 65° C., about 70° C., about 75° C. or about 80° C. is used.

In some embodiments, crystallization will be complete within about 24 to about 72 hours, but longer and shorter periods are possible depending on the choice of crystallizing solvent and temperature.

The precipitation and/or crystallization of the ethanesulfonic acid salt, in some embodiments, is carried out by filtering the salt from solution. In other embodiments of the invention, the precipitation and/or crystallization is induced by the addition of anti-solvent. A suitable anti-solvent can contain any solvent in which the salt is poorly soluble such as a ketone (e.g., methyl isobutyl ketone).

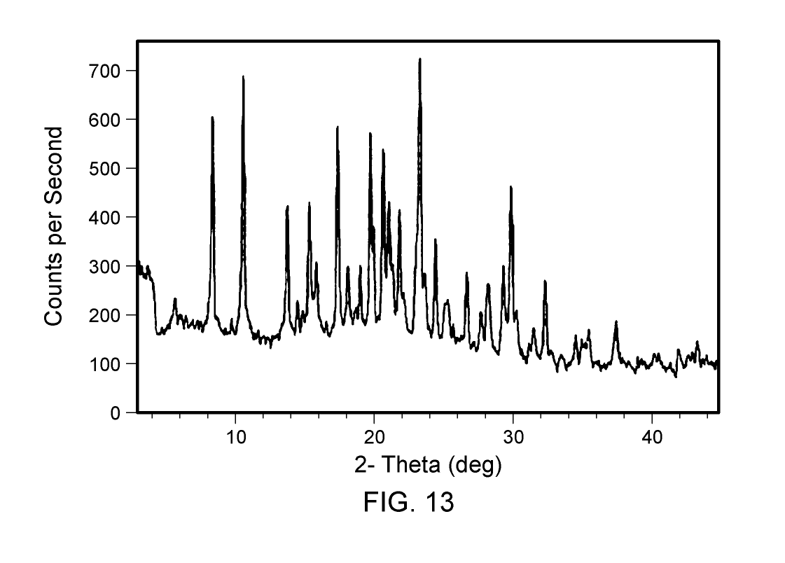

The crystalline ethanesulfonic acid salt of the compound of Formula I can be identified by its unique signatures with respect to, for example, X-ray powder diffraction, differential scanning calorimetry (DSC), thermogravimetric analysis (TGA), and solid state NMR. In some embodiments, the crystalline ethanesulfonic acid salt can be characterized by the X-ray powder diffraction (XRPD) pattern substantially as shown in FIG. 13. Major peaks from the)(RFD are listed below in Table 5. The term “substantially” in this instance indicates that 2-theta values for individual peaks can vary by about ±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline ethanesulfonic acid salt form of the compound of Formula I has an XRPD pattern having at least 3 peaks, in terms of 2θ, selected from Table 5 (CPS less than 250=“+;” CPS of 250 to 500=“++;” CPS greater than 500=“+++”). In some embodiments, the ethanesulfonic acid salt form has an XRPD pattern having at least 3 peaks, in terms of 2θ, selected from about 4.1°, about 8.4°, about 10.6°, about 13.7°, about 14.5°, about 15.4°, about 15.8°, about 17.4°, about 18.1°, about 19.0°, about 19.8°, about 20.7°, about 21.0°, about 21.9°, or about 23.3°. In some embodiments, the ethanesulfonic acid salt has an XRPD pattern having at least one peak, in terms of 2θ, at about 8.4°. In some embodiments, the ethanesulfonic acid salt has an XRPD pattern having peaks, in terms of 2θ, at about 8.4°, about 10.6°, and about 17.4°. In some embodiments, the ethanesulfonic acid salt has an XRPD pattern having peaks, in terms of 2θ, at about 8.4°, about 10.6°, about 17.4°, and about 19.8°.

TABLE 5

| Observed Peak, | Intensity |
|---|---|
| 2θ (°) | (CPS) |
| 4.1 | + |
| 8.4 | ++ |
| 10.6 | +++ |
| 13.7 | ++ |
| 14.5 | + |
| 15.4 | ++ |
| 15.8 | + |
| 17.4 | ++ |
| 18.1 | + |
| 19.0 | + |
| 19.8 | ++ |
| 20.7 | ++ |
| 21.0 | + |
| 21.9 | + |
| 23.3 | +++ |
| 23.6 | + |
| 24.4 | + |
| 25.2 | + |
| 26.6 | + |
| 27.7 | + |
| 28.2 | + |
| 29.3 | + |
| 29.8 | ++ |
| 31.3 | + |
| 32.2 | + |
| 34.4 | + |
| 35.0 | + |
| 35.3 | + |
| 37.3 | + |
| 41.8 | + |

### Methods

In some embodiments, salts of the compound of Formula I can modulate activity of one or more of various kinases including, for example, phosphoinositide 3-kinases (PI3Ks). The term “modulate” is meant to refer to an ability to increase or decrease the activity of one or more members of the PI3K family. Accordingly, the salts of the invention can be used in methods of modulating a PI3K by contacting the PI3K with any one or more of the salts or compositions described herein. In some embodiments, salts of the present invention can act as inhibitors of one or more PI3Ks. In further embodiments, salts of the invention can be used to modulate activity of a PI3K in an individual in need of modulation of the receptor by administering a modulating amount of salts of the invention. In some embodiments, modulating is inhibiting.

Given that cancer cell growth and survival is impacted by multiple signaling pathways, the present invention is useful for treating disease states characterized by drug resistant kinase mutants. In addition, different kinase inhibitors, exhibiting different preferences in the kinases which they modulate the activities of, may be used in combination. This approach could prove highly efficient in treating disease states by targeting multiple signaling pathways, reducing the likelihood of drug-resistance arising in a cell, and reducing the toxicity of treatments for disease.

Kinases to which the present salts bind and/or modulate (e.g., inhibit) include any member of the PI3K family. In some embodiments, the PI3K is PI3Kα, PI3Kβ, PI3Kγ, or PI3Kδ. In some embodiments, the PI3K is PI3Kγ or PI3Kδ. In some embodiments, the PI3K is PI3Kγ. In some embodiments, the PI3K is PI3Kδ. In some embodiments, the PI3K includes a mutation. A mutation can be a replacement of one amino acid for another, or a deletion of one or more amino acids. In such embodiments, the mutation can be present in the kinase domain of the PI3K.

In some embodiments, more than one salts of the invention is used to inhibit the activity of one kinase (e.g., PI3Kγ or PI3Kδ).

In some embodiments, more than one salts of the invention is used to inhibit more than one kinase, such as at least two kinases (e.g., PI3Kγ and PI3Kδ).

In some embodiments, one or more of the salts is used in combination with another kinase inhibitor to inhibit the activity of one kinase (e.g., PI3Kγ or PI3Kδ).

In some embodiments, one or more of the salts is used in combination with another kinase inhibitor to inhibit the activities of more than one kinase (e.g., PI3Ky or PI3Kδ), such as at least two kinases.

The salts of the invention can be selective. By “selective” is meant that the salts binds to or inhibits a kinase with greater affinity or potency, respectively, compared to at least one other kinase. In some embodiments, the salts of the invention are selective inhibitors of PI3Kγ or PI3Kδ over PI3Kα and/or PI3Kβ. In some embodiments, the salts of the invention are selective inhibitors of PI3Kδ (e.g., over PI3Kα, PI3Kβ and PI3Kγ). In some embodiments, the salts of the invention are selective inhibitors of PI3Kγ (e.g., over PI3Kα, PI3Kβ and PI3Kδ). In some embodiments, selectivity can be at least about 2-fold, 5-fold, 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the Kₘ ATP concentration of each enzyme. In some embodiments, the selectivity of salts of the invention can be determined by cellular assays associated with particular PI3K kinase activity.

Another aspect of the present invention pertains to methods of treating a kinase (such as PI3K)-associated disease or disorder in an individual (e.g., patient) by administering to the individual in need of such treatment a therapeutically effective amount or dose of one or more salts of the present invention or a pharmaceutical composition thereof. A PI3K-associated disease can include any disease, disorder or condition that is directly or indirectly linked to expression or activity of the PI3K, including overexpression and/or abnormal activity levels. In some embodiments, the disease can be linked to Akt (protein kinase B), mammalian target of rapamycin (mTOR), or phosphoinositide-dependent kinase 1 (PDK1). In some embodiments, the mTOR-related disease can be inflammation, atherosclerosis, psoriasis, restenosis, benign prostatic hypertrophy, bone disorders, pancreatitis, angiogenesis, diabetic retinopathy, arthritis, immunological disorders, kidney disease, or cancer. A PI3K-associated disease can also include any disease, disorder or condition that can be prevented, ameliorated, or cured by modulating PI3K activity. In some embodiments, the disease is characterized by the abnormal activity of PI3K. In some embodiments, the disease is characterized by mutant PI3K. In such embodiments, the mutation can be present in the kinase domain of the PI3K.

Examples of PI3K-associated diseases include immune-based diseases involving the system including, for example, rheumatoid arthritis, allergy, asthma, glomerulonephritis, lupus, or inflammation related to any of the above.

Further examples of PI3K-associated diseases include cancers such as breast, prostate, colon, endometrial, brain, bladder, skin, uterus, ovary, lung, pancreatic, renal, gastric, or hematological cancer.

In some embodiments, the hematological cancer is acute myeloblastic leukemia (AML) or chronic myeloid leukemia (CIVIL), or B cell lymphoma.

Further examples of PI3K-associated diseases include lung diseases such as acute lung injury (ALI) and adult respiratory distress syndrome (ARDS).

Further examples of PI3K-associated diseases include osteoarthritis, restenosis, atherosclerosis, bone disorders, arthritis, diabetic retinopathy, psoriasis, benign prostatic hypertrophy, inflammation, angiogenesis, pancreatitis, kidney disease, inflammatory bowel disease, myasthenia gravis, multiple sclerosis, or Sjoegren's syndrome, and the like.

Further examples of PI3K-associated diseases include idiopathic thrombocytopenic purpura (ITP), autoimmune hemolytic anemia (AIHA), vasculitis, systemic lupus erythematosus, lupus nephritis, pemphigus, membranous nephropathy, chronic lymphocytic leukemia (CLL), Non-Hodgkin lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, or germinal center B cell (GCB) diffuse large B cell lymphoma.

In some embodiments, the present application provides a method of treating pemphigus, membranous nephropathy, Hodgkin's lymphoma, Waldenstrom's macroglobulinemia, prolymphocytic leukemia, acute lymphoblastic leukemia, myelofibrosis, mucosa-associated lymphatic tissue (MALT) lymphoma, mediastinal (thymic) large B-cell lymphoma, lymphomatoid granulomatosis, splenic marginal zone lymphoma, primary effusion lymphoma, intravascular large B-cell lymphoma, plasma cell leukemia, extramedullary plasmacytoma, smouldering myeloma (aka asymptomatic myeloma), or monoclonal gammopathy of undetermined significance (MGUS).

In some embodiments, the present application provides a method of treating osteoarthritis, restenosis, atherosclerosis, bone disorders, arthritis, diabetic retinopathy, psoriasis, benign prostatic hypertrophy, inflammation, angiogenesis, pancreatitis, kidney disease, inflammatory bowel disease, myasthenia gravis, multiple sclerosis, or Sjögren's syndrome.

In some embodiments, the disease is idiopathic thrombocytopenic purpura (ITP), autoimmune hemolytic anemia (AIHA), vasculitis, pemphigus, or membranous nephropathy.

In some embodiments, the idiopathic thrombocytopenic purpura (ITP) is selected from relapsed ITP and refractory ITP.

In some embodiments, the vasculitis is selected from Behcet's disease, Cogan's syndrome, giant cell arteritis, polymyalgia rheumatica (PMR), Takayasu's arteritis, Buerger's disease (thromboangiitis obliterans), central nervous system vasculitis, Kawasaki disease, polyarteritis nodosa, Churg-Strauss syndrome, mixed cryoglobulinemia vasculitis (essential or hepatitis C virus (HCV)-induced), Henoch-Schönlein purpura (HSP), hypersensitivity vasculitis, microscopic polyangiitis, Wegener's granulomatosis, and anti-neutrophil cytoplasm antibody associated (ANCA) systemic vasculitis (AASV).

In some embodiments, the present application provides methods of treating an immune-based disease, cancer, or lung disease in a patient.

In some embodiments, the immune-based disease is systemic lupus erythematosus or lupus nephritis.

In some embodiments, the cancer is breast cancer, prostate cancer, colon cancer, endometrial cancer, brain cancer, bladder cancer, skin cancer, cancer of the uterus, cancer of the ovary, lung cancer, pancreatic cancer, renal cancer, gastric cancer, or a hematological cancer.

In some embodiments, the hematological cancer is acute myeloblastic leukemia, chronic myeloid leukemia, B cell lymphoma, chronic lymphocytic leukemia (CLL), Non-Hodgkins lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, or germinal center B cell (GCB) diffuse large B cell lymphoma.

In some embodiments, the non-Hodgkin lymphoma (NHL) is selected from relapsed NHL, refractory NHL, and recurrent follicular NHL.

In some embodiments, the lung disease is acute lung injury (ALI) or adult respiratory distress syndrome (ARDS).

The present application further provides a salt described herein for use in any of the methods described herein.

The present application further provides use of a salt described herein for the production of a medicament for use in any of the methods described herein.

As used herein, the term “contacting” refers to the bringing together of indicated moieties in an in vitro system or an in vivo system. For example, “contacting” a PI3K with a salt of the invention includes the administration of a salt of the present invention to an individual or patient, such as a human, having a PI3K, as well as, for example, introducing a salt of the invention into a sample containing a cellular or purified preparation containing the PI3K.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active salts or pharmaceutical agent that elicits the biological or medicinal response that is being sought in a tissue, system, animal, individual or human by a researcher, veterinarian, medical doctor or other clinician. In some embodiments, the dosage of the salts, administered to a patient or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Combination Therapies

One or more additional pharmaceutical agents such as, for example, chemotherapeutics, anti-inflammatory agents, steroids, immunosuppressants, as well as Bcr-Abl, Flt-3, EGFR, HER2, JAK (e.g., JAK1 or JAK2), c-MET, VEGFR, PDGFR, cKit, IGF-1R, RAF, FAK,Akt mTOR, PIM, and AKT (e.g., AKT1, AKT2, or AKT3) kinase inhibitors such as, for example, those described in WO 2006/056399, or other agents such as, therapeutic antibodies can be used in combination with the salts of the present invention for treatment of PI3K-associated diseases, disorders or conditions. The one or more additional pharmaceutical agents can be administered to a patient simultaneously or sequentially. In some embodiments, the additional pharmaceutical agent is a JAK1 and/or JAK2 inhibitor. In some embodiments, the present application provides a method of treating a disease described herein (e.g., a B cell malignancy, such as diffuse B-cell lymphoma) in a patient comprising administering to the patient a compound described herein, or a pharmaceutically acceptable salt thereof, and a JAK1 and/or JAK2 inhibitor. The B cell malignancies can include those described herein and in U.S. Ser. No. 61/976,815, filed Apr. 8, 2014. In some embodiments, the inhibitor of JAK1 and/or JAK2 is 3-cyclopentyl-3-[4-(7H-pyrrolo[2,3-d]pyrimidin-4-yl)-1H-pyrazol-1-yl]propanenitrile. In some embodiments, the inhibitor of JAK1 and/or JAK2 is (3R)-3-cyclopentyl-3-[4-(7H-pyrrolo[2,3-d]pyrimidin-4-yl)-1H-pyrazol-1-yl]propanenitrile (ruxolitinib; also known as INCB018424). Ruxolitinib has an IC₅₀ of less than 10 nM at 1 mM ATP (Assay G) at JAK1 and JAK2. 3-Cyclopentyl-3-[4-(7H-pyrrolo[2,3-d]pyrimidin-4-yl)-1H-pyrazol-1-yl]propanenitrile and ruxolitinib can be made by the procedure described in U.S. Pat. No. 7,598,257 (Example 67), filed Dec. 12, 2006, which is incorporated herein by reference in its entirety. In some embodiments, the inhibitor of JAK1 and/or JAK2 is (3R)-3-cyclopentyl-3-[4-(7H-pyrrolo[2,3-d]pyrimidin-4-yl)-1H-pyrazol-1-yl]propanenitrile phosphoric acid salt.

In some embodiments, the inhibitor of JAK1 and/or JAK2 is a compound of Table A, or a pharmaceutically acceptable salt thereof. The compounds in Table A are selective JAK1 inhibitors (selective over JAK2, JAK3, and TYK2). The IC₅₀ obtained by the method of Assay G at 1 mM ATP are shown in Table A.

TABLE A

| | | | JAK1 | |
|---|---|---|---|---|
| | | | IC₅₀ | JAK2/ |
| # | Name/Reference | Structure | (nM) | JAK1 |
| 1 | ((2R,5S)-5-{2-[(1R)-1- hydroxyethyl]-1H-imidazo[4,5- d]thieno[3,2-b]pyridin-1- yl}tetrahydro-2H-pyran-2- yl)acetonitrile US 2014/0121198, Example 20 | 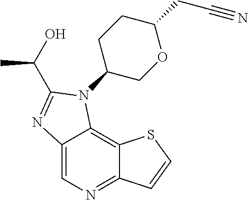 [SMILES: C[C@@H](O)c1nc2cnc3ccsc3c2n1[C@H]1CC[C@H](CC#N)OC1] | ++ | >10 |
| 2 | 4-[3-(cyanomethyl)-3-(3′,5′- dimethyl-1H,1′H-4,4′-bipyrazol-1- yl)azetidin-1-yl]-2,5-difluoro-N- [(1S)-2,2,2-trifluoro-1- methylethyl]benzamide US 2014/0343030, Example 7 | 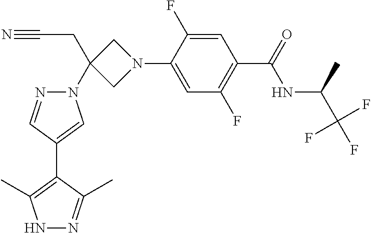 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 3 | 3-[1-(6-chloropyridin-2- yl)pyrrolidin-3-yl]-3-[4-(7H- pyrrolo[2,3-d]pyrimidin-4-yl)-1H- pyrazol-1-yl]propanenitrile US 2010/0298334 Example 2^{a} | 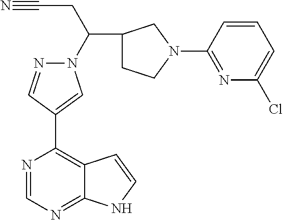 [SMILES: N#CCC(C1CCN(c2cccc(Cl)n2)C1)n1cc(-c2ncnc3[nH]ccc23)cn1] | + | >10 |
| 4 | 3-(1-[1,3]oxazolo[5,4-b]pyridin-2- ylpyrrolidin-3-yl)-3-[4-(7H- pyrrolo[2,3-d]pyrimidin-4-yl)-1H- pyrazol-1-yl]propanenitrile US 2010/0298334 (Example 13c) | 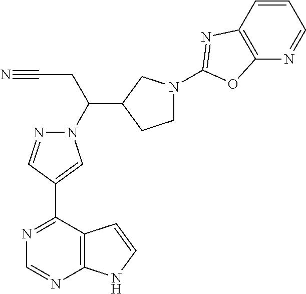 [SMILES: N#CCC(C1CCN(c2nc3cccnc3o2)C1)n1cc(-c2ncnc3[nH]ccc23)cn1] | + | >10 |
| 5 | 4-[(4-{3-cyano-2-[4-(7H- pyrrolo[2,3-d]pyrimidin-4-yl)-1H- pyrazol-1-yl]propyl}piperazin-1- yl)carbonyl]-3-fluorobenzonitrile US 2011/0059951 (Example 12) | 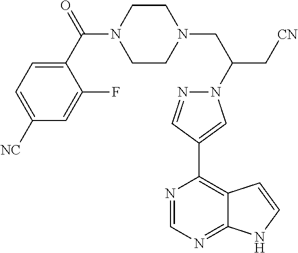 [SMILES: N#CCC(CN1CCN(C(=O)c2ccc(C#N)cc2F)CC1)n1cc(-c2ncnc3[nH]ccc23)cn1] | + | >10 |
| 6 | 4-[(4-{3-cyano-2-[3-(7H- pyrrolo[2,3-d]pyrimidin-4-yl)-1H- pyrrol-1-yl]propyl}piperazin-1- yl)carbonyl]-3-fluorobenzonitrile US 2011/0059951 (Example 13) | 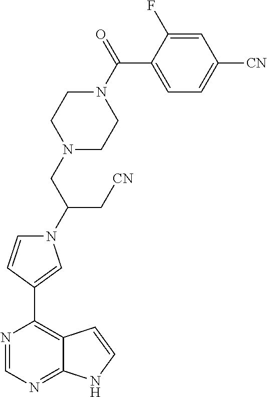 [SMILES: N#CCC(CN1CCN(C(=O)c2ccc(C#N)cc2F)CC1)n1ccc(-c2ncnc3[nH]ccc23)c1] | + | >10 |
| 7 | {1-{1-[3-Fluoro-2- (trifluoromethyl)isonicotinoyl] piperidin-4-yl}-3-[4-(7H-pyrrolo[2,3- d]pyrimidin-4-yl)-1H-pyrazol-1- yl]azetidin-3-yl}acetonitrile US 2011/0224190 (Example 1) | 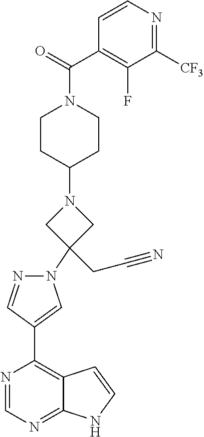 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 8 | 4-{3-(Cyanomethyl)-3-[4-(7H- pyrrolo[2,3-d]pyrimidin-4-yl)-1H- pyrazol-1-yl]azetidin-1-yl}-N-[4- fluoro-2- (trifluoromethyl)phenyl]piperidine- 1-carboxamide US 2011/0224190 (Example 154) | 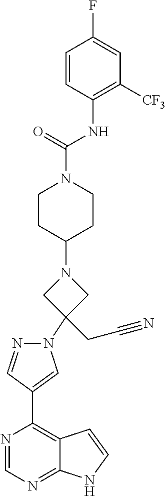 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 9 | [3-[4-(7H-pyrrolo[2,3- d]pyrimidin-4-yl)-1H-pyrazol-1- yl]-1-(1-{[2- (trifluoromethyl)pyrimidin-4- yl]carbonyl}piperidin-4- yl)azetidin-3-yl]acetonitrile US 2011/0224190 (Example 85) | 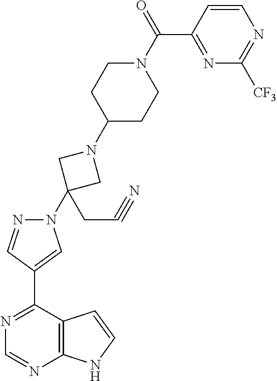 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 10 | [trans-1-[4-(7H-pyrrolo[2,3- d]pyrimidin-4-yl)-1H-pyrazol-1- yl]-3-(4-{[2- (trifluoromethyl)pyrimidin-4- yl]carbonyl}piperazin-1- yl)cyclobutyl]acetonitrile US 2012/0149681 (Example 7b) | 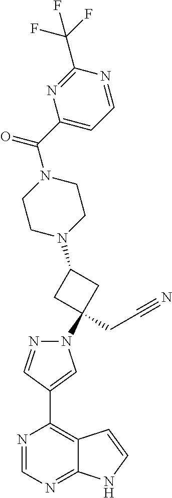 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | {trans-3-(4-{[4-[(3- hydroxyazetidin-1-yl)methyl]-6- (trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2012/0149681 (Example 157) | 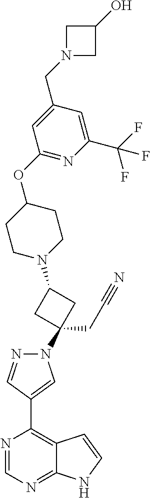 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | {trans-3-(4-{[4-{[(2S)-2- (hydroxymethyl)pyrrolidin-1- yl]methyl}-6- (trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2012/0149681 (Example 161) | 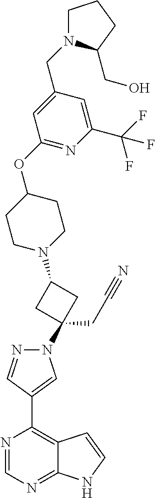 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | {trans-3-(4-{[4-{[(2R)-2- (hydroxymethyl)pyrrolidin-1- yl]methyl}-6- (trifluoromethyl)pyridin-2- yl]oxy}piperidin-1-yl)-1-[4-(7H- pyrrolo[2,3-d]pyrimidin-4-yl)-1H- pyrazol-1- yl]cyclobutyl}acetonitrile US 2012/0149681 (Example 162) | 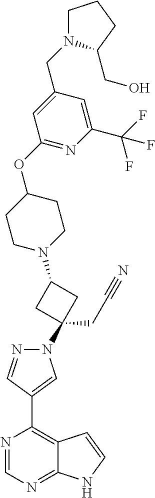 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | 4-(4-{3-[(dimethylamino)methyl]- 5-fluorophenoxy}piperidin-1-yl)- 3-[4-(7H-pyrrolo[2,3-d]pyrimidin- 4-yl)-1H-pyrazol-1- yl]butanenitrile US 2012/0149682 (Example 20)^{b} | 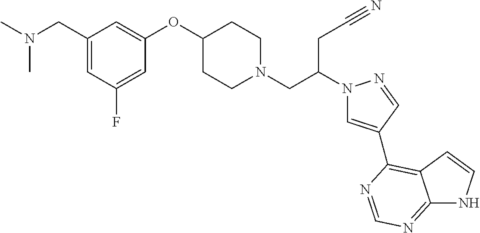 [SMILES: CN(C)Cc1cc(F)cc(OC2CCN(CC(CC#N)n3cc(-c4ncnc5[nH]ccc45)cn3)CC2)c1] | + | >10 |
| 15 | 5-{3-(cyanomethyl)-3-[4-(7H- pyrrolo[2,3-d]pyrimidin-4-yl)-1H- pyrazol-1-yl]azetidin-1-yl}-N- isopropylpyrazine-2-carboxamide US 2013/0018034 (Example 18) | 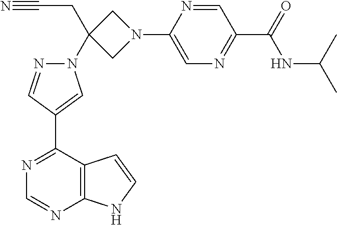 [SMILES: CC(C)NC(=O)c1cnc(N2CC(CC#N)(n3cc(-c4ncnc5[nH]ccc45)cn3)C2)cn1] | + | >10 |
| 16 | 4-{3-(cyanomethyl)-3-[4-(7H- pyrrolo[2,3-d]pyrimidin-4-yl)-1H- pyrazol-1-yl]azetidin-1-yl}-2,5- difluoro-N-[(1S)-2,2,2-trifluoro-1- methylethyl]benzamide US 2013/0018034 (Example 28) | 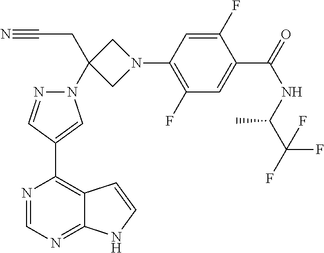 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | 5-{3-(cyanomethyl)-3-[4-(1H- pyrrolo[2,3-b]pyridin-4-yl)-1H- pyrazol-1-yl]azetidin-1-yl}-N- isopropylpyrazine-2-carboxamide US 2013/0018034 (Example 34) | 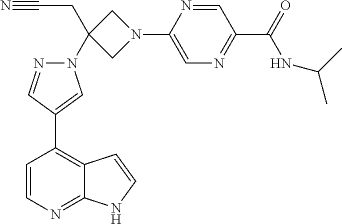 [SMILES: CC(C)NC(=O)c1cnc(N2CC(CC#N)(n3cc(-c4ccnc5[nH]ccc45)cn3)C2)cn1] | + | >10 |
| 18 | {1-(cis-4-{[6-(2-hydroxyethyl)-2- (trifluoromethyl)pyrimidin-4- yl]oxy}cyclohexyl)-3-[4-(7H- pyrrolo[2,3-d]pyrimidin-4-yl)-1H- pyrazol-1-yl]azetidin-3- yl}acetonitrile US 2013/0045963 (Example 45) | 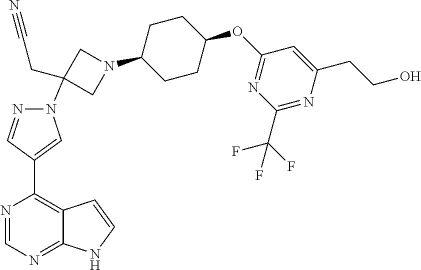 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | {1-(cis-4-{[4- [(ethylamino)methyl]-6- (trifluoromethyl)pyridin-2- yl]oxy}cyclohexyl)-3-[4-(7H- pyrrolo[2,3-d]pyrimidin-4-yl)-1H- pyrazol-1-yl]azetidin-3- yl}acetonitrile US 2013/0045963 (Example 65) | 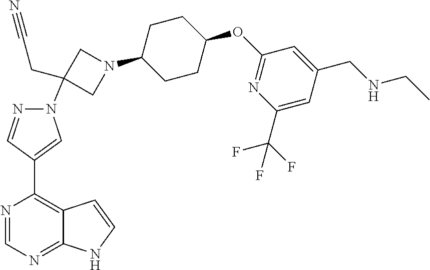 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | {1-(cis-4-{[4-(1-hydroxy-1- methylethyl)-6- (trifluoromethyl)pyridin-2- yl]oxy}cyclohexyl)-3-[4-(7H- pyrrolo[2,3-d]pyrimidin-4-yl)-1H- pyrazol-1-yl]azetidin-3- yl}acetonitrile US 2013/0045963 (Example 69) | 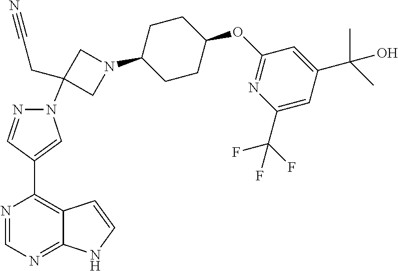 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | {1-(cis-4-{[4-{[(3R)-3- hydroxypyrrolidin-1-yl]methyl}-6- (trifluoromethyl)pyridin-2- yl]oxy}cyclohexyl)-3-[4-(7H- pyrrolo[2,3-d]pyrimidin-4-yl)-1H- pyrazol-1-yl]azetidin-3- yl}acetonitrile US 2013/0045963 (Example 95) | 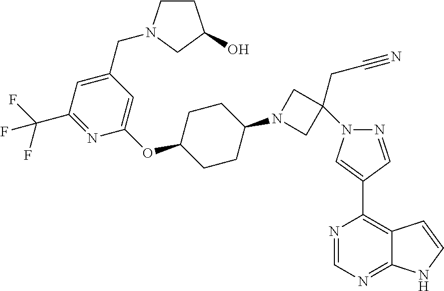 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | {1-(cis-4-{[4-{[(3S)-3- hydroxypyrrolidin-1-yl]methyl}-6- (trifluoromethyl)pyridin-2- yl]oxy}cyclohexyl)-3-[4-(7H- pyrrolo[2,3-d]pyrimidin-4-yl)-1H- pyrazol-1-yl]azetidin-3- yl}acetonitrile US 2013/0045963 (Example 95) | 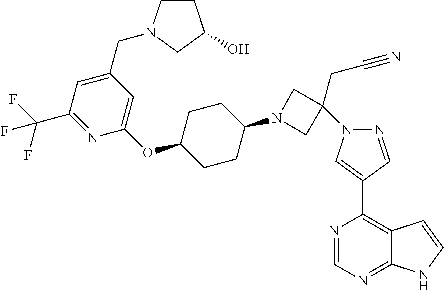 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | {trans-3-(4-{[4-({[(1S)-2- hydroxy-1- methylethyl]amino}methyl)-6- (trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2014/0005166 (Example 1) | 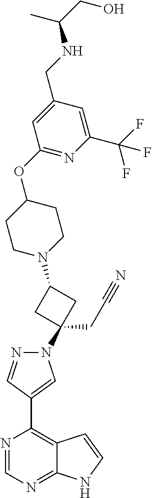 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | {trans-3-(4-{[4-({[(2R)-2- hydroxypropyl]amino}methyl)-6- (trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2014/0005166 (Example 14) | 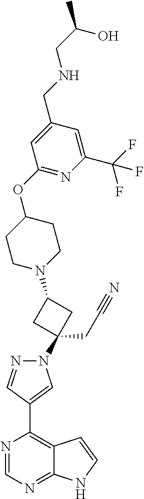 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | {trans-3-(4-{[4-({[(2S)-2- hydroxypropyl]amino}methyl)-6- (trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2014/0005166 (Example 15) | 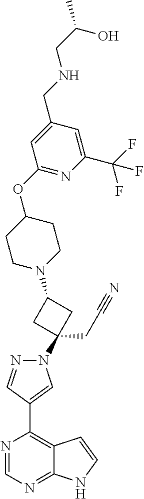 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | {trans-3-(4-{[4-(2-hydroxyethyl)- 6-(trifluoromethyl)pyridin-2- yl]oxy}piperidin-1-yl)-1-[4-(7H- pyrrolo[2,3-d]pyrimidin-4-yl)-1H- pyrazol-1-yl]cyclobutyl}acetonitrile US 2014/0005166 (Example 20) | 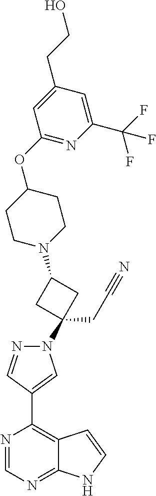 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example G for assay conditions)
++ means ≦100 nM (see Example G for assay conditions)
+++ means ≦300 nM (see Example G for assay conditions)
^{a}Data for enantiomer 1
^{b}Data for enantiomer 2

In some embodiments, the inhibitor of JAK1 and/or JAK2 is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the inhibitor of JAK1 and/or JAK2 is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

In some embodiments, the inhibitor of JAK1 and/or JAK2 is 4-{3-(cyanomethyl)-3-[4-(7H-pyrrolo[2,3-d]pyrimidin-4-yl)-1H-pyrazol-1-yl]azetidin-1-yl}-2,5-difluoro-N-[(1S)-2,2,2-trifluoro-1-methylethyl]benzamide, or a pharmaceutically acceptable salt thereof.

In some embodiments, the inhibitor of JAK1 and/or JAK2 is selected from (R)-3-[1-(6-chloropyridin-2-yl)pyrrolidin-3-yl]-3-[4-(7H-pyrrolo[2,3-d]pyrimidin-4-yl)-1H-pyrazol-1-yl]propanenitrile, (R)-3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile, (R)-4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile, (R)-4-[(4-{3 -cyano-2-[3 -(7H-pyrrolo[2,3 -d]pyrimidin-4-yl)-1H-pyrrol-1-yl]propyl}piperazin-1-yl)carbonyl]-3-fluorobenzonitrile, or (R)-4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile, (S)-3-[1-(6-chloropyridin-2-yl)pyrrolidin-3-yl]-3-[4-(7H-pyrrolo[2,3-d]pyrimidin-4-yl)-1H-pyrazol-1-yl]propanenitrile, (S)-3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile, (S)-4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile, (S)-4-[(4-{3-cyano-2-[3-(7H-pyrrolo[2,3-d]pyrimidin-4-yl)-1H-pyrrol-1-yl]propyl}piperazin-1-yl)carbonyl]-3-fluorobenzonitrile, (S)-4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile; and pharmaceutically acceptable salts of any of the aforementioned.

In some embodiments, the compounds of Table A are prepared by the synthetic procedures described in US Patent Publ. No. 2010/0298334, filed May 21, 2010, US Patent Publ. No. 2011/0059951, filed Aug. 31, 2010, US Patent Publ. No. 2011/0224190, filed Mar. 9, 2011, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the inhibitor of JAK1 and/or JAK2 is selected from the compounds of US Patent Publ. No. 2010/0298334, filed May 21, 2010, US Patent Publ. No. 2011/0059951, filed Aug. 31, 2010, US Patent Publ. No. 2011/0224190, filed Mar. 9, 2011, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

Example antibodies for use in combination therapy include but are not limited to trastuzumab (e.g. anti-HER2), ranibizumab (e.g. anti-VEGF-A), bevacizumab (trade name avastin, e.g. anti-VEGF, panitumumab (e.g. anti-EGFR), cetuximab (e.g. anti-EGFR), rituxan (anti-CD20) and antibodies directed to c-MET.

One or more of the following agents may be used in combination with the salts of the present invention and are presented as a non limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa, Tarceva, antibodies to EGFR, Gleevec™, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17.alpha.-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225, Campath, Clofarabine, cladribine, aphidicolon, rituxan, sunitinib, dasatinib, tezacitabine, Smll, fludarabine, pentostatin, triapine, didox, trimidox, amidox, 3-AP, MDL-101,731, bendamustine (Treanda), ofatumumab, and GS-1101 (also known as CAL-101).

Example chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include compounds, and their pharmaceutically acceptable salts, as disclosed in WO 03/037347, WO 03/099771, and WO 04/046120.

Example suitable RAF inhibitors include compounds, and their pharmaceutically acceptable salts, as disclosed in WO 00/09495 and WO 05/028444.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable mTOR inhibitors include compounds, and their pharmaceutically acceptable salts, as disclosed in WO 2011/025889.

In some embodiments, the salts of the invention can be used in combination with one or more other kinase inhibitors including imatinib, particularly for treating patients resistant to imatinib or other kinase inhibitors.

In some embodiments, the salts of the invention can be used in combination with a chemotherapeutic in the treatment of cancer, such as multiple myeloma, and may improve the treatment response as compared to the response to the chemotherapeutic agent alone, without exacerbation of its toxic effects. Examples of additional pharmaceutical agents used in the treatment of multiple myeloma, for example, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. Additive or synergistic effects are desirable outcomes of combining a PI3K inhibitor of the present invention with an additional agent. Furthermore, resistance of multiple myeloma cells to agents such as dexamethasone may be reversible upon treatment with the PI3K inhibitor of the present invention. The agents can be combined with the present salts in a single or continuous dosage form, or the agents can be administered simultaneously or sequentially as separate dosage forms.

In some embodiments, a corticosteroid such as dexamethasone is administered to a patient in combination with the salts of the invention where the dexamethasone is administered intermittently as opposed to continuously.

In some further embodiments, combinations of the salts of the invention with other therapeutic agents can be administered to a patient prior to, during, and/or after a bone marrow transplant or stem cell transplant.

Pharmaceutical Formulations and Dosage Forms

When employed as pharmaceuticals, the salts of the compound of Formula I can be administered in the form of pharmaceutical compositions. These compositions can be prepared in a manner well known in the pharmaceutical art, and can be administered by a variety of routes depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable. This invention also includes pharmaceutical compositions which contain, as the active ingredient, one or more of the salts of the compound of Formula I in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active salt, soft and hard gelatin capsules, suppositories, sterile injectable solutions, and sterile packaged powders.

In preparing a formulation, the active salt can be milled to provide the appropriate particle size prior to combining with the other ingredients. If the active salt is substantially insoluble, it can be milled to a particle size of less than 200 mesh. If the active salt is substantially water soluble, the particle size can be adjusted by milling to provide a substantially uniform distribution in the formulation, e.g. about 40 mesh.

The salts of the invention may be milled using known milling procedures such as wet milling to obtain a particle size appropriate for tablet formation and for other formulation types. Finely divided (nanoparticulate) preparations of the salts of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the invention contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the invention contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the invention contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the salts described herein in the methods and uses of the invention.

The active salt can be effective over a wide dosage range and is generally administered in a pharmaceutically effective amount. It will be understood, however, that the amount of the salt actually administered will usually be determined by a physician, according to the relevant circumstances, including the condition to be treated, the chosen route of administration, the actual salt administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a salt of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the salts and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the salt of the invention. The topical formulations can be suitably packaged in tubes of, for example, 100 g which are optionally associated with instructions for the treatment of the select indication, e.g., psoriasis or other skin condition.

The amount of salt or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the salt preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the salts of the present invention can vary according to, for example, the particular use for which the treatment is made, the manner of administration of the salt, the health and condition of the patient, and the judgment of the prescribing physician. The proportion or concentration of a salt of the invention in a pharmaceutical composition can vary depending upon a number of factors including dosage, chemical characteristics (e.g., hydrophobicity), and the route of administration. For example, the salts of the invention can be provided in an aqueous physiological buffer solution containing about 0.1 to about 10% w/v of the salt for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the salt selected, formulation of the excipient, and its route of administration. Effective doses can be extrapolated from dose-response curves derived from in vitro or animal model test systems.

The salts of the invention can further include one or more additional pharmaceutical agents such as a chemotherapeutic, steroid, anti-inflammatory compound, or immunosuppressant, examples of which are listed herein.

### Labeled Salts and Assay Methods

Another aspect of the present invention relates to labeled salts of the invention (radio-labeled, fluorescent-labeled, etc.) that would be useful not only in imaging techniques but also in assays, both in vitro and in vivo, for localizing and quantitating PI3K enzyme in tissue samples, including human, and for identifying ligands by inhibition binding of a labeled salt. Accordingly, the present application includes PI3K assays that contain such labeled salts.

The present application further includes isotopically-labled salts of the invention. An “isotopically” or “radio-labeled” salt is a salt of the invention where one or more atoms are replaced or substituted by an atom having an atomic mass or mass number different from the atomic mass or mass number typically found in nature (i.e., naturally occurring). Suitable radionuclides that may be incorporated in salts of the present application include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled salts will depend on the specific application of that radio-labeled salt. For example, for in vitro PI3K labeling and competition assays, salts that incorporate ³H, ¹⁴C, ⁸²Br, ¹²⁵I, ¹³¹I, ³⁵S or will generally be most useful. For radio-imaging applications ¹¹C, ¹⁸, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled salt” is a salt that has incorporated at least one radionuclide. In some embodiments the radionuclide is selected from the group consisting of ³H, ¹⁴C, ¹²⁵I, ³⁵S and ⁸²Br. In some embodiments, one or more H atoms for any salt described herein is each replaced by a deuterium atom.

The present application can further include synthetic methods for incorporating radio-isotopes into compounds of the invention. Synthetic methods for incorporating radio-isotopes into organic compounds are well known in the art, and an ordinary skill in the art will readily recognize the methods applicable for the salts of invention.

A labeled salt of the invention can be used in a screening assay to identify/evaluate salts. For example, a newly synthesized or identified salt (i.e., test salt) which is labeled can be evaluated for its ability to bind a PI3K by monitoring its concentration variation when contacting with the PI3K, through tracking of the labeling. For example, a test salt (labeled) can be evaluated for its ability to reduce binding of another compound or salt which is known to bind to a PI3K (i.e., standard compound). Accordingly, the ability of a test salt to compete with the standard compound for binding to the PI3K directly correlates to its binding affinity. Conversely, in some other screening assays, the standard compound is labeled and test salts are unlabeled. Accordingly, the concentration of the labeled standard compound is monitored in order to evaluate the competition between the standard compound and the test salt, and the relative binding affinity of the test salt is thus ascertained.

### Kits

The present invention also includes pharmaceutical kits useful, for example, in the treatment or prevention of PI3K-associated diseases or disorders, such as cancer, which include one or more containers containing a pharmaceutical composition comprising a therapeutically effective amount of a salt of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Experimental Methods

In the below examples, X-Ray Powder Diffraction analysis was carried out on a Rigaku MiniFlex X-ray Powder Diffractometer (XRPD) instrument with the following parameters: radiation source is Cu at 1.054056 Å with K_{β} filter and X-ray power of 30 KV, 15 mA. The sample powder was dispersed on a zero-background sample holder. General measurement conditions were:
Start Angle-3°
Stop Angle-45°
Sampling-0.02 deg.
Scan speed-2 deg/min.

Differential Scanning calorimetry (DSC) was carried out on a TA Instrument Differential Scanning calorimetry, Model Q20 with autosampler. The general experimental conditions were: 30-260° C. at 10° C./min, nitrogen gas flow at 50 mL/min, using an aluminum sample pan.

Thermogravimetric analysis (TGA) was carried out on a TA Instrument Thermogravimetric Analyzer, Model Q500 with the following conditions: Ramp at 20° C./min. to 600° C.; nitrogen gas at 40 mL/min balance purge flow; 60 mL/min sample purge flow; and platinum sample pan.

### Example 2

### Preparation of Adipic Acid Salt

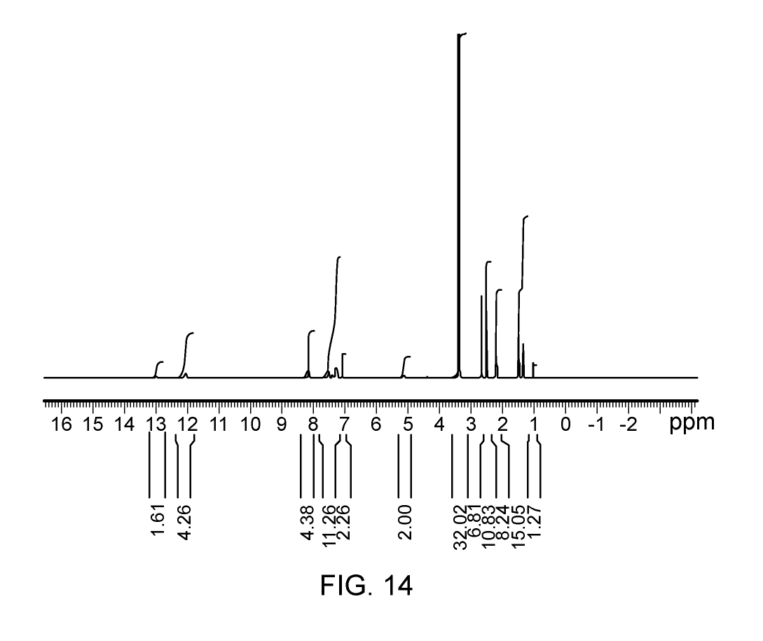

Adipic acid (46.99 mg, 0.322 mmol, 1.36 eq) was added to a solution of (S)-7-(1-(9H-purin-6-ylamino)ethyl)-6-(3-fluorophenyl)-3-methyl-5H-thiazolo[3,2-a]pyrimidin-5-one (99.96 mg, 0.237 mmol) in 2-propanol (2.3 mL) with stirring at room temperature. A slurry was formed and the reaction mixture was heated to 65° C. to give a clear solution. The solution was cooled to room temperature to give a thick slurry, which was then stirred at room temperature for 4 h. The solid was collected by filtration, washed with heptane (3 mL) and dried at room temperature under vacuum overnight to provide (S)-7-(1-(9H-purin-6-ylamino)ethyl)-6-(3-fluorophenyl)-3-methyl-5H-thiazolo[3,2-a]pyrimidin-5-one adipate (130.8 mg, 97.1%) as an off-white solid. (400 MHz, d6-DMSO). The stoichiometric ratio of the salt between the free base and the adipic acid was determined to be 1:1 by ¹H NMR (400 MHz, d₆-DMSO). See FIG. 14.

Analytical data collected on the product, including characterization by XRPD, DSC, and TGA were performed as described in Example 1. XRFD, DSC, and TGA spectra of the adipic acid salt are provided in FIGS. 1-3, respectively. DSC data indicates that the adipic acid salt has an onset temperature of 176° C. and a peak at 178° C. TGA data shows a 0.037%-weight loss up to about 100° C.

### Example 3

### Preparation of Hydrochloric Acid Salt

Hydrochloric acid (0.31 mL, 1 M in 2-propanol, 0.31 mmol, 1.3 eq.) was added to a solution of (S)-7-(1-(9H-purin-6-ylamino)ethyl)-6-(3-fluorophenyl)-3-methyl-5H-thiazolo[3,2-a]pyrimidin-5-one (100.9 mg, 0.237 mmol) dissolved in 2-propanol (2.7 mL). The resulting slurry was stirred at room temperature for 30 min and heated at 65° C. for 30 min. The solution was then cooled to room temperature and stirred for 4 h. The solid was collected by filtration, washed with heptane and dried to yield (S)-7-(1-(9H-purin-6-ylamino)ethyl)-6-(3-fluorophenyl)-3-methyl-5H-thiazolo[3,2-a]pyrimidin-5-one hydrochloride (107.1 mg, 97.6%).

Analytical data collected on the product, including characterization by XRFD, DSC, and TGA were performed as described in Example 1. XRPD, DSC, and TGA spectra of the hydrochloric acid salt are provided in FIGS. 4-6, respectively. The stoichiometric ratio of the salt between the free base and hydrochloric acid was determined to be 1:1 by HPLC with an Evaporative Light Scattering Detector. DSC data indicates that the hydrochloride has an onset temperature of 168° C. and a peak at 183° C. TGA data shows a 0.056% weight loss up to about 100° C.

### Example 4

### Preparation of Methanesulfonic Acid Salt

### Method 1

To a solution of methanesulfonic acid in isopropyl alcohol (0.500 M, 0.076 mL, 0.038 mmol, 1.070 eq) was added 0.250 mL of the solution of (S)-7-(1-(9H-purin-6-ylamino)ethyl)-6-(3-fluorophenyl)-3-methyl-5H-thiazolo[3,2-a]pyrimidin-5-one in isopropyl alcohol (0.142 M, 0.0355 mmol, 1 eq) followed by stirring for 4 h, to which was added 0.2 mL of heptane to give a thin slurry. The reaction mixture was heated for 20 min at 66-68° C. and cooled to room temperature. The slurry was filtered and the solid was dried under vacuum to afford (S)-7-(1-(9H-purin-6-ylamino)ethyl)-6-(3-fluorophenyl)-3-methyl-5H-thiazolo[3,2-a]pyrimidin-5-one methanesulfonic acid.

### Method 2

A reactor was charged with (S)-7-(1-(9H-purin-6-ylamino)ethyl)-6-(3-fluorophenyl)-3-methyl-5H-thiazolo[3,2-a]pyrimidin-5-one (102.3 mg, 0.243 mmol) and 2-propanol (2.3 mL), followed by addition of methanesulfonic acid in 2-propanol (0.31 mL, 0.31 mmol, 1 M in 2-propanol) to give a clear solution. Seeds were added and the solution was stirred for 20 min to form a slurry. The reaction mixture was then stirred at 70° C. for 20 min, cooled to room temperature and stirred for 4 h. The solid was filtered, washed with heptane (3 mL) and dried overnight at room temperature under vacuum to provide (S)-7-(1-(9H-purin-6-ylamino)ethyl)-6-(3-fluorophenyl)-3-methyl-5H-thiazolo[3,2-a]pyrimidin-5-one mesylate (118.8 mg, 96.7%) as an off-white solid.

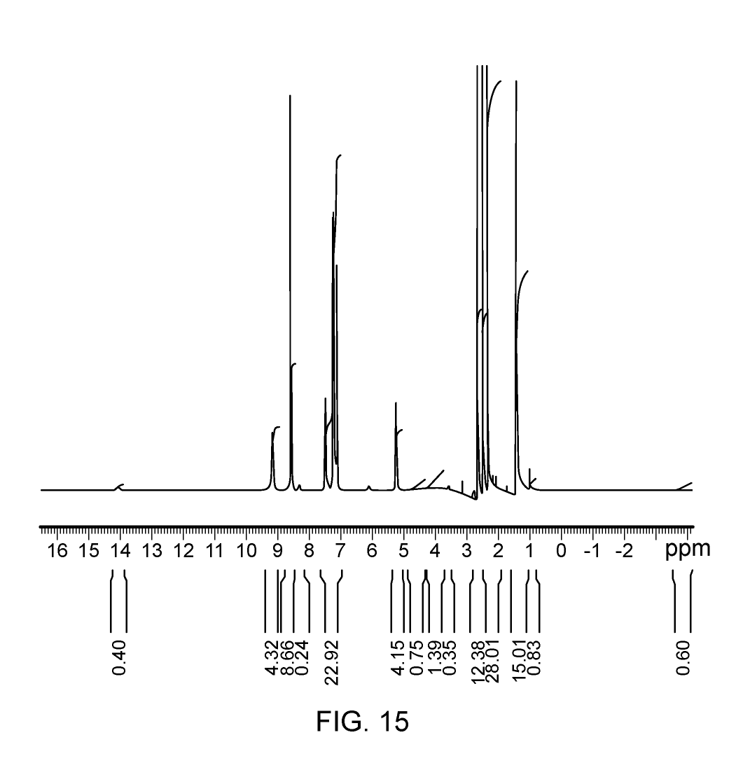

Analytical data collected on the product, including characterization by XRFD, DSC, and TGA, were performed as described in Example 1. XRPD, DSC, and TGA spectra of the methanesulfonic acid salt are provided in FIGS. 7-9, respectively. The stoichiometric ratio of the salt between the free base and methanesulfonic acid was determined to be 1:1 by ¹H NMR (400 MHz, d₆-DMSO). See FIG. 15. DSC data indicates that the mesylate has an onset temperature of 206° C. and a peak at 212° C. TGA data shows a 0.056% weight loss up to about 100° C.

### Example 5

### Preparation of Gentisic Acid Salt

To a flask was added (S)-7-(1-(9H-purin-6-ylamino)ethyl)-6-(3-fluorophenyl)-3-methyl-5H-thiazolo[3,2-a]pyrimidin-5-one (102.66 mg, 0.244 mmol), and 2-propanol (2 mL) to give a clear solution, followed by addition of gentisic acid (48.8 mg, 0.317 mmol). The mixture was stirred for 30 min to form a slurry and then heated to 65° C. to give a clear solution. The solution was cooled to room temperature and stirred for 4 h. The solid was collected by filtration, washed with heptane and dried overnight under vacuum to yield (S)-7-(1-(9H-purin-6-ylamino)ethyl)-6-(3-fluorophenyl)-3-methyl-5H-thiazolo[3,2-a]pyrimidin-5-one gentisate (109.7 mg, 80%) as off-white solid.

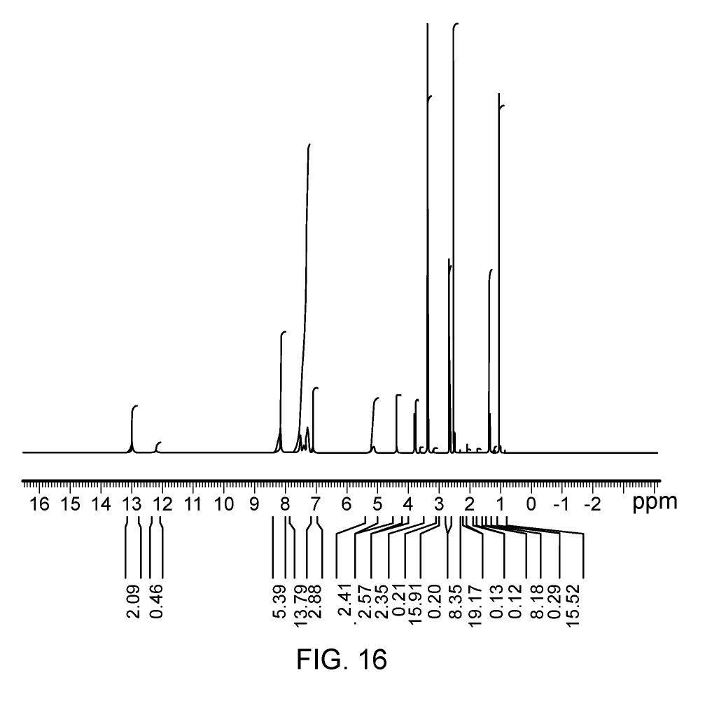

Analytical data collected on the product, including characterization by XRFD, DSC, and TGA were performed as described in Example 1. XRPD, DSC, and TGA spectra of the gentisic acid salt are provided in FIGS. 10-12, respectively. TGA data shows a 0.059% weight loss up to about 100° C. ¹H NMR data (400 MHz, d₆-DMSO) is provided in FIG. 16.

### Example 6

### Preparation of Ethanesulfonic Acid Salt

To a solution of ethanesulfonic acid in isopropyl alcohol (0.1 M, 0.36 mL, 0.036 mmol, 1.014 eq) was added 0.25 mL of the solution of (S)-7-(1-(9H-purin-6-ylamino)ethyl)-6-(3-fluorophenyl)-3-methyl-5H-thiazolo[3,2-a]pyrimidin-5-one in isopropyl alcohol (0.142 M, 0.0355 mmol, 1 eq) followed by stirring for 4 h to give good solid, which was filtered and dried under vacuum to afford (S)-7-(1-(9H-purin-6-ylamino)ethyl)-6-(3-fluorophenyl)-3-methyl-5H-thiazolo[3,2-a]pyrimidin-5-one ethanesulfonic acid.

Analytical data, including XRPD data, was performed as described in Example 1. The XRPD spectrum of the ethanesulfonic acid salt is provided in FIG. 13.

### Example A

### PI3K Enzyme Assay

PI3-Kinase luminescent assay kit including lipid kinase substrate, D-myo-phosphatidylinositol 4,5-bisphosphate (PtdIns(4,5)P2)D (+)-sn-1,2-di-O-octanoylglyceryl, 3-O-phospho linked (PIP2), biotinylated I(1,3,4,5)P4, PI(3,4,5)P3 Detector Protein is purchased from Echelon Biosciences (Salt Lake City, UT). AlphaScreen™ GST Detection Kit including donor and acceptor beads is purchased from PerkinElmer Life Sciences (Waltham, Mass.). PI3Kδ (p110δ/p85α) is purchased from Millipore (Bedford, Mass.). ATP, MgCl₂, DTT, EDTA, HEPES and CHAPS are purchased from Sigma-Aldrich (St. Louis, Mo.).

### AlphaScreen™ Assay for PI3Kδ

The kinase reactions are conducted in 384-well REMP plate from Thermo Fisher Scientific in a final volume of 40 μL. Inhibitors are first diluted serially in DMSO and added to the plate wells before the addition of other reaction components. The final concentration of DMSO in the assay is 2%. The PI3K assays are carried out at room temperature in 50 mM HEPES, pH 7.4, 5 mM MgCl₂, 50 mM NaCl, 5 mM DTT and CHAPS 0.04%. Reactions are initiated by the addition of ATP, the final reaction mixture of 20 μM PIP2, 20 μM ATP, 1.2 nM PI3Kδ is incubated for 20 minutes. 10 μL of reaction mixture are then transferred to 5 μL 50 nM biotinylated I(1,3,4,5)P4 in quench buffer: 50 mM HEPES pH 7.4, 150 mM NaCl, 10 mM EDTA, 5 mM DTT, 0.1% Tween-20, followed with the addition of 10 μL AlphaScreen™ donor and acceptor beads suspended in quench buffer containing 25 nM PI(3,4,5)P3 detector protein. The final concentration of both donor and acceptor beads is 20 mg/ml. After plate sealing, the plate is incubated in a dark location at room temperature for 2 hours. The activity of the product is determined on Fusion-alpha microplate reader (Perkin-Elmer). IC₅₀ determination is performed by fitting the curve of percent control activity versus the log of the inhibitor concentration using the GraphPad Prism 3.0 software.

### Example B

### PI3K Enzyme Assay

Materials: Lipid kinase substrate, phosphoinositol-4,5-bisphosphate (PIP2), are purchased from Echelon Biosciences (Salt Lake City, Utah). PI3K isoforms α, β, δ and γ are purchased from Millipore (Bedford, Mass.). ATP, MgC12, DTT, EDTA, MOPS and CHAPS are purchased from Sigma-Aldrich (St. Louis, Mo.).

The kinase reaction are conducted in clear-bottom 96-well plate from Thermo Fisher Scientific in a final volume of 24 μL. Inhibitors are first diluted serially in DMSO and added to the plate wells before the addition of other reaction components. The final concentration of DMSO in the assay is 0.5%. The PI3K assays are carried out at room temperature in 20 mM MOPS, pH 6.7, 10 mM MgCl₂, 5 mM DTT and CHAPS 0.03%. The reaction mixture is prepared containing 50 μM PIP2, kinase and varying concentration of inhibitors. Reactions are initiated by the addition of ATP containing 2.2 μCi [y-³³P]ATP to a final concentration of 1000 μM. The final concentration of PI3K isoforms α, β, δ and γ in the assay are 1.3, 9.4, 2.9 and 10.8 μM, respectively. Reactions are incubated for 180 minutes and terminated by the addition of 100 μL of 1 M potassium phosphate pH 8.0, 30 mM EDTA quench buffer. A 100 μL aliquot of the reaction solution are then transferred to 96-well Millipore MultiScreen IP 0.45 μm PVDF filter plate (The filter plate is prewetted with 200 μL 100% ethanol, distilled water, and 1 M potassium phosphate pH 8.0, respectively). The filter plate is aspirated on a Millipore Manifold under vacuum and washed with 18×200 μL wash buffer containing 1 M potassium phosphate pH 8.0 and 1 mM ATP. After drying by aspiration and blotting, the plate is air dried in an incubator at 37° C. overnight. Packard TopCount adapter (Millipore) is then attached to the plate followed with addition of 120 μL Microscint 20 scintillation cocktail (Perkin Elmer) in each well. After the plate sealing, the radioactivity of the product is determined by scintillation counting on Topcount (Perkin-Elmer). IC₅₀ determination is performed by fitting the curve of percent control activity versus the log of the inhibitor concentration using the GraphPad Prism 3.0 software.

### Example C

### PI3Kδ Scintillation Proximity Assay

### Materials

[γ-³³P]ATP (10 mCi/mL) is purchased from Perkin-Elmer (Waltham, Mass.). Lipid kinase substrate, D-myo-Phosphatidylinositol 4,5-bisphosphate (PtdIns(4,5)P2)D (+)-sn-1,2-di-O-octanoylglyceryl, 3-O-phospho linked (PIP2), CAS 204858-53-7, are purchased from Echelon Biosciences (Salt Lake City, Utah). PI3Kδ (p110δ/p85α) was purchased from Millipore (Bedford, Mass.). ATP, MgCl₂, DTT, EDTA, MOPS and CHAPS are purchased from Sigma-Aldrich (St. Louis, Mo.). Wheat Germ Agglutinin (WGA) YSi SPA Scintillation Beads are purchased from GE healthcare life sciences (Piscataway, N.J.).

The kinase reaction is conducted in polystyrene 384-well matrix white plate from Thermo Fisher Scientific in a final volume of 25 μL. Inhibitors are first diluted serially in DMSO and added to the plate wells before the addition of other reaction components. The final concentration of DMSO in the assay is 0.5%. The PI3K assays are carried out at room temperature in 20 mM MOPS, pH 6.7, 10 mM MgCl₂, 5 mM DTT and CHAPS 0.03%. Reactions are initiated by the addition of ATP, the final reaction mixture consists of 20 μM PIP2, 20 μM ATP, 0.2 μCi [γ-³³P] ATP, 4 nM PI3Kδ. Reactions are incubated for 210 min and terminated by the addition of 40 μL SPA beads suspended in quench buffer: 150 mM potassium phosphate pH 8.0, 20% glycerol, 25 mM EDTA, 400 μM ATP. The final concentration of SPA beads is 1.0 mg/mL. After the plate sealing, plates are shaken overnight at room temperature and centrifuged at 1800 rpm for 10 minutes, the radioactivity of the product is determined by scintillation counting on Topcount (Perkin-Elmer). IC₅₀ determination is performed by fitting the curve of percent control activity versus the log of the inhibitor concentration using the GraphPad Prism 3.0 software.

### Example D

### B Cell Proliferation Assay

To acquire B cells, human PBMC are isolated from the peripheral blood of normal, drug free donors by standard density gradient centrifugation on Ficoll-Hypague (GE Healthcare, Piscataway, N.J.) and incubated with anti-CD19 microbeads (Miltenyi Biotech, Auburn, Calif.). The B cells are then purified by positive immunosorting using an autoMacs (Miltenyi Biotech) according to the manufacture's instruction.

The purified B cells (2×10⁵/well/200 μL) are cultured in 96-well ultra-low binding plates (Corning, Corning, N.Y.) in RPMI1640, 10% FBS and goat F(ab′)2 anti-human IgM (10 μg/mL) (Invitrogen, Carlsbad, Calif.) in the presence of different amount of test salts for three days. [³H]-thymidine (1 μCi/well) (PerkinElmer, Boston, Mass.) in PBS is then added to the B cell cultures for an additional 12 hours before the incorporated radioactivity is separated by filtration with water through GF/B filters (Packard Bioscience, Meriden, Conn.) and measured by liquid scintillation counting with a TopCount (Packard Bioscience).

### Example E

### Pfeiffer Cell Proliferation Assay

Pfeiffer cell line (diffuse large B cell lymphoma) are purchased from ATCC (Manassas, Va.) and maintained in the culture medium recommended (RPMI and 10% FBS). To measure the anti-proliferation activity of the salts, the Pfeiffer cells are plated with the culture medium (2×10³ cells/well/per 200 μl) into 96-well ultra-low binding plates (Corning, Corning, N.Y.), in the presence or absence of a concentration range of test salts. After 3-4 days, [³H]-thymidine (1 μCi/well) (PerkinElmer, Boston, Mass.) in PBS is then added to the cell culture for an additional 12 hours before the incorporated radioactivity is separated by filtration with water through GF/B filters (Packard Bioscience, Meridenj, Conn.) and measured by liquid scintillation counting with a TopCount (Packard Bioscience).

### Example F

### Akt Phosphorylation Assay

Ramos cells (B lymphocyte from Burkitts lymphoma) are obtained from ATCC (Manassas, Va.) and maintained in RPMI1640 and 10% FBS. The cells (3×10⁷ cells /tube/3 mL in RPMI) are incubated with different amounts of test salts for 2 hrs at 37° C. and then stimulated with goat F(ab′)2 anti-human IgM (5 μg/mL) (Invitrogen) for 17 minutes in a 37° C. water bath. The stimulated cells are spun down at 4° C. with centrifugation and whole cell extracts are prepared using 300 μL lysis buffer (Cell Signaling Technology, Danvers, Mass.). The resulting lysates are sonicated and supernatants are collected. The phosphorylation level of Akt in the supernatants are analyzed by using PathScan phospho-Akt1 (Ser473) sandwich ELISA kits (Cell Signaling Technology) according to the manufacturer's instruction.

### Example G

### In vitro JAK Kinase Assay

The compounds in Table A were tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) were expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC_{50S} of compounds were measured for each kinase in the 40 μL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions was 1 mM. Reactions were carried out at room temperature for 1 hour and then stopped with 20 μL 45 mM EDTA, 300 nM SA-APC, 6 nM Eu-Py20 in assay buffer (Perkin Elmer, Boston, Mass.). Binding to the Europium labeled antibody took place for 40 minutes and HTRF signal was measured on a PHERA star plate reader (BMG, Cary, N.C.). The data for the JAK1 and/or JAK2 inhibitors were obtained by testing the compounds in the Example G assay at 1 mM ATP.

Various modifications of the invention, in addition to those described herein, will be apparent to those skilled in the art from the foregoing description. Such modifications are also intended to fall within the scope of the appended claims. Each reference, including patents, patent applications, and publications, cited in the present application is incorporated herein by reference in its entirety.

What is claimed is:

1. A pharmaceutically acceptable salt form of a compound of Formula I: 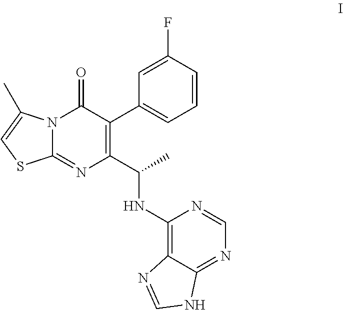 [SMILES: Cc1csc2nc([C@H](C)Nc3ncnc4[nH]cnc34)c(-c3cccc(F)c3)c(=O)n12]
wherein the salt is an adipic acid salt, a hydrochloric acid salt, a methanesulfonic acid salt, a gentisic acid salt, or an ethanesulfonic acid salt.

2. The salt form of claim 1 which is an adipic acid salt.

3. The salt form of claim 1 which is a hydrochloric acid salt.

4. The salt form of claim 1 which is a methanesulfonic acid salt.

5. The salt form of claim 1 which is a gentisic acid salt.

6. The salt form of claim 1 which is an ethanesulfonic acid salt.

7. The salt form of claim 1 which is crystalline.

8. The salt form of claim 1 which is anhydrous.

9. The salt form of claim 1 which is substantially isolated.

10. A composition comprising the salt form of claim 1 and a pharmaceutically acceptable carrier.

11. A method of treating a disease in a patient, wherein the disease is associated with abnormal expression or activity of a PI3K kinase, comprising administering to said patient a therapeutically effective amount of a salt form of claim 1.

12. The method of claim 11, wherein the disease is osteoarthritis, restenosis, atherosclerosis, bone disorders, arthritis, diabetic retinopathy, psoriasis, benign prostatic hypertrophy, inflammation, angiogenesis, pancreatitis, kidney disease, inflammatory bowel disease, myasthenia gravis, multiple sclerosis, or Sjogren's syndrome.

13. The method claim 11, wherein more than one of the salts is administered.

14. The method of claim 11, wherein the salt is administered in combination with a kinase inhibitor that inhibits a kinase other than a PI3K kinase.

15. A method of treating an immune-based disease in a patient, comprising administering to the patient a therapeutically effective amount of a salt form of claim 1.

16. The method of claim 15, wherein said immune-based disease is rheumatoid arthritis, allergy, asthma, glomerulonephritis, lupus, or inflammation related to any of the aforementioned.

17. A method of treating a cancer in a patient, comprising administering to the patient a therapeutically effective amount of a salt form of claim 1.

18. The method of claim 17, wherein the cancer is breast, prostate, colon, endometrial, brain, bladder, skin, uterus, ovary, lung, pancreatic, renal, gastric, or a hematological cancer.

19. The method of claim 18, wherein the hematological cancer is acute myeloblastic leukemia, chronic myeloid leukemia, B cell lymphoma, chronic lymphocytic leukemia (CLL), Non-Hodgkins lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, or germinal center B cell (GCB) diffuse large B cell lymphoma.

20. A method of treating a lung disease in a patient, comprising administering to said patient a therapeutically effective amount of a salt form of claim 1.

21. The method of claim 20, wherein said lung disease is acute lung injury (ALI) or adult respiratory distress syndrome (ARDS).

22. A method of preparing a salt form of claim 1 comprising combining the compound of Formula I with adipic acid, hydrochloric acid, methanesulfonic acid, gentisic acid, or ethanesulfonic acid.
